SEQUENCE LISTING (154 sequences)

SEQ ID NO: 1 - DNA, 15 bp, Homo sapiens
gctagacgttagcgt

SEQ ID NO: 2 - DNA, 15 bp, Homo sapiens
gctagatgttagcgt

SEQ ID NO: 3 - DNA, 15 bp, Homo sapiens
gctagacgttagcgt

SEQ ID NO: 4 - DNA, 15 bp, Homo sapiens
gctagacgttagcgt

SEQ ID NO: 5 - DNA, 15 bp, Homo sapiens
gcatgacgttgagct

SEQ ID NO: 6 - DNA, 20 bp, Homo sapiens
atggaaggtccagcgttctc

SEQ ID NO: 7 - DNA, 20 bp, Homo sapiens
atcgactctcgagcgttctc

SEQ ID NO: 8 - DNA, 20 bp, Homo sapiens
atcgactctcgagcgttctc

SEQ ID NO: 9 - DNA, 20 bp, Homo sapiens
atcgactctcgagcgttctc

SEQ ID NO: 10 - DNA, 20 bp, Homo sapiens
atggaaggtccaacgttctc

SEQ ID NO: 11 - DNA, 20 bp, Homo sapiens
gagaacgctggaccttccat

SEQ ID NO: 12 - DNA, 20 bp, Homo sapiens
gagaacgctcgaccttccat

SEQ ID NO: 13 - DNA, 20 bp, Homo sapiens
gagaacgctcgaccttcgat

SEQ ID NO: 14 - DNA, 20 bp, Homo sapiens
gagaacgctggaccttccat

SEQ ID NO: 15 - DNA, 20 bp, Homo sapiens
gagaacgatggaccttccat

SEQ ID NO: 16 - DNA, 20 bp, Homo sapiens
gagaacgctccagcactgat

SEQ ID NO: 17 - DNA, 20 bp, Homo sapiens
tccatgtcggtcctgatgct

SEQ ID NO: 18 - DNA, 20 bp, Homo sapiens
tccatgtcggtcctgatgct

SEQ ID NO: 19 - DNA, 20 bp, Homo sapiens
tccatgacgttcctgatgct

SEQ ID NO: 20 - DNA, 20 bp, Homo sapiens
tccatgtcggtcctgctgat

SEQ ID NO: 21 - DNA, 8 bp, Homo sapiens
tcaacgtt

SEQ ID NO: 22 - DNA, 8 bp, Homo sapiens
tcagcgct

SEQ ID NO: 23 - DNA, 8 bp, Homo sapiens
tcatcgat

SEQ ID NO: 24 - DNA, 8 bp, Homo sapiens
tcttcgaa

SEQ ID NO: 25 - DNA, 7 bp, Homo sapiens
caacgtt

SEQ ID NO: 26 - DNA, 8 bp, Homo sapiens
ccaacgtt

SEQ ID NO: 27 - DNA, 8 bp, Homo sapiens
aacgttct

SEQ ID NO: 28 - DNA, 8 bp, Homo sapiens
tcaacgtc

SEQ ID NO: 29 - DNA, 20 bp, Homo sapiens
atggactctccagcgttctc

SEQ ID NO: 30 - DNA, 20 bp, Homo sapiens
atggaaggtccaacgttctc

SEQ ID NO: 31 - DNA, 20 bp, Homo sapiens
atcgactctcgagcgttctc

SEQ ID NO: 32 - DNA, 20 bp, Homo sapiens
atggaggctccatcgttctc

SEQ ID NO: 33 - DNA, 20 bp, Homo sapiens
atcgactctcgagcgttctc

SEQ ID NO: 34 - DNA, 20 bp, Homo sapiens
atcgactctcgagcgttctc

SEQ ID NO: 35 - DNA, 20 bp, Homo sapiens
tccatgtcggtcctgatgct

SEQ ID NO: 36 - DNA, 20 bp, Homo sapiens
tccatgccggtcctgatgct

SEQ ID NO: 37 - DNA, 20 bp, Homo sapiens
tccatggcggtcctgatgct

SEQ ID NO: 38 - DNA, 20 bp, Homo sapiens
tccatgacggtcctgatgct

SEQ ID NO: 39 - DNA, 20 bp, Homo sapiens
tccatgtcgatcctgatgct

SEQ ID NO: 40 - DNA, 20 bp, Homo sapiens
tccatgtcgctcctgatgct

SEQ ID NO: 41 - DNA, 20 bp, Homo sapiens
tccatgtcgtccctgatgct

SEQ ID NO: 42 - DNA, 20 bp, Homo sapiens
tccatgacgtgcctgatgct

SEQ ID NO: 43 - DNA, 20 bp, Homo sapiens
tccataacgttcctgatgct

SEQ ID NO: 44 - DNA, 20 bp, Homo sapiens
tccatgacgtccctgatgct

SEQ ID NO: 45 - DNA, 20 bp, Homo sapiens
tccatcacgtgcctgatgct

SEQ ID NO: 46 - DNA, 19 bp, Homo sapiens
ggggtcaacgttgacgggg

SEQ ID NO: 47 - DNA, 19 bp, Homo sapiens
ggggtcagtcgtgacgggg

SEQ ID NO: 48 - DNA, 15 bp, Homo sapiens
gctagacgttagtgt

SEQ ID NO: 49 - DNA, 20 bp, Homo sapiens
tccatgtcgttcctgatgct

SEQ ID NO: 50 - DNA, 24 bp, Homo sapiens
accatggacgatctgtttcccctc

SEQ ID NO: 51 - DNA, 18 bp, Homo sapiens
tctcccagcgtgcgccat

SEQ ID NO: 52 - DNA, 24 bp, Homo sapiens
accatggacgaactgtttcccctc

SEQ ID NO: 53 - DNA, 24 bp, Homo sapiens
accatggacgagctgtttcccctc

SEQ ID NO: 54 - DNA, 24 bp, Homo sapiens
accatggacgacctgtttcccctc

SEQ ID NO: 55 - DNA, 24 bp, Homo sapiens
accatggacgtactgtttcccctc

SEQ ID NO: 56 - DNA, 24 bp, Homo sapiens
accatggacggtctgtttcccctc

SEQ ID NO: 57 - DNA, 24 bp, Homo sapiens
accatggacgttctgtttcccctc

SEQ ID NO: 58 - DNA, 15 bp, Homo sapiens
cacgttgaggggcat

SEQ ID NO: 59 - DNA, 12 bp, Homo sapiens
tcagcgtgcgcc

SEQ ID NO: 60 - DNA, 17 bp, Homo sapiens
atgacgttcctgacgtt

SEQ ID NO: 61 - DNA, 17 bp, Homo sapiens
tctcccagcgggcgcat

SEQ ID NO: 62 - DNA, 20 bp, Homo sapiens
tccatgtcgttcctgtcgtt

SEQ ID NO: 63 - DNA, 20 bp, Homo sapiens
tccatagcgttcctagcgtt

SEQ ID NO: 64 - DNA, 21 bp, Homo sapiens
tcgtcgctgtctccccttctt

SEQ ID NO: 65 - DNA, 19 bp, Homo sapiens
tcctgacgttcctgacgtt

SEQ ID NO: 66 - DNA, 19 bp, Homo sapiens
tcctgtcgttcctgtcgtt

SEQ ID NO: 67 - DNA, 20 bp, Homo sapiens
tccatgtcgtttttgtcgtt

SEQ ID NO: 68 - DNA, 20 bp, Homo sapiens
tcctgtcgttccttgtcgtt

SEQ ID NO: 69 - DNA, 20 bp, Homo sapiens
tccttgtcgttcctgtcgtt

SEQ ID NO: 70 - DNA, 20 bp, Homo sapiens
tcctgtcgttttttgtcgtt

SEQ ID NO: 71 - DNA, 21 bp, Homo sapiens
tcgtcgctgtctgcccttctt

SEQ ID NO: 72 - DNA, 21 bp, Homo sapiens
tcgtcgctgttgtcgtttctt

SEQ ID NO: 73 - DNA, 20 bp, Homo sapiens
tccatgcgtgcgtgcgtttt

SEQ ID NO: 74 - DNA, 20 bp, Homo sapiens
tccatgcgttgcgttgcgtt

SEQ ID NO: 75 - DNA, 20 bp, Homo sapiens
tccacgacgttttcgacgtt

SEQ ID NO: 76 - DNA, 20 bp, Homo sapiens
tcgtcgttgtcgttgtcgtt

SEQ ID NO: 77 - DNA, 24 bp, Homo sapiens
tcgtcgttttgtcgttttgtcgtt

SEQ ID NO: 78 - DNA, 22 bp, Homo sapiens
tcgtcgttgtcgttttgtcgtt

SEQ ID NO: 79 - DNA, 21 bp, Homo sapiens
gcgtgcgttgtcgttgtcgtt

SEQ ID NO: 80 - DNA, 21 bp, Homo sapiens
tgtcgtttgtcgtttgtcgtt

SEQ ID NO: 81 - DNA, 25 bp, Homo sapiens
tgtcgttgtcgttgtcgttgtcgtt

SEQ ID NO: 82 - DNA, 19 bp, Homo sapiens
tgtcgttgtcgttgtcgtt

SEQ ID NO: 83 - DNA, 14 bp, Homo sapiens
tcgtcgtcgtcgtt

SEQ ID NO: 84 - DNA, 13 bp, Homo sapiens
tgtcgttgtcgtt

SEQ ID NO: 85 - DNA, 20 bp, Homo sapiens
tccatagcgttcctagcgtt

SEQ ID NO: 86 - DNA, 20 bp, Homo sapiens
tccatgacgttcctgacgtt

SEQ ID NO: 87 - DNA, 6 bp, Homo sapiens
gtcgyt

SEQ ID NO: 88 - DNA, 7 bp, Homo sapiens
tgtcgyt

SEQ ID NO: 89 - DNA, 18 bp, Homo sapiens
agctatgacgttccaagg

SEQ ID NO: 90 - DNA, 20 bp, Homo sapiens
tccatgacgttcctgacgtt

SEQ ID NO: 91 - DNA, 20 bp, Homo sapiens
atcgactctcgaacgttctc

SEQ ID NO: 92 - DNA, 20 bp, Homo sapiens
tccatgtcggtcctgacgca

SEQ ID NO: 93 - DNA, 8 bp, Homo sapiens
tcttcgat

SEQ ID NO: 94 - DNA, 20 bp, Homo sapiens
ataggaggtccaacgttctc

SEQ ID NO: 95 - DNA, 15 bp, Homo sapiens
gctagaggggagggt

SEQ ID NO: 96 - DNA, 15 bp, Homo sapiens
gctagatgttagggg

SEQ ID NO: 97 - DNA, 15 bp, Homo sapiens
gctagaggggagggt

SEQ ID NO: 98 - DNA, 15 bp, Homo sapiens
gctagaggggagggt

SEQ ID NO: 99 - DNA, 15 bp, Homo sapiens
gcatgagggggagct

SEQ ID NO: 100 - DNA, 20 bp, Homo sapiens
atggaaggtccagggggctc

SEQ ID NO: 101 - DNA, 20 bp, Homo sapiens
atggactctggagggggctc

SEQ ID NO: 102 - DNA, 20 bp, Homo sapiens
atggactctggagggggctc

SEQ ID NO: 103 - DNA, 20 bp, Homo sapiens
atggactctggagggggctc

SEQ ID NO: 104 - DNA, 20 bp, Homo sapiens
atggaaggtccaaggggctc

SEQ ID NO: 105 - DNA, 20 bp, Homo sapiens
gagaaggggggaccttccat

SEQ ID NO: 106 - DNA, 20 bp, Homo sapiens
gagaaggggggaccttccat

SEQ ID NO: 107 - DNA, 20 bp, Homo sapiens
gagaaggggggaccttggat

SEQ ID NO: 108 - DNA, 20 bp, Homo sapiens
gagaaggggggaccttccat

SEQ ID NO: 109 - DNA, 20 bp, Homo sapiens
gagaaggggggaccttccat

SEQ ID NO: 110 - DNA, 20 bp, Homo sapiens
gagaaggggccagcactgat

SEQ ID NO: 111 - DNA, 20 bp, Homo sapiens
tccatgtggggcctgatgct

SEQ ID NO: 112 - DNA, 20 bp, Homo sapiens
tccatgtggggcctgatgct

SEQ ID NO: 113 - DNA, 20 bp, Homo sapiens
tccatgaggggcctgatgct

SEQ ID NO: 114 - DNA, 20 bp, Homo sapiens
tccatgtggggcctgctgat

SEQ ID NO: 115 - DNA, 20 bp, Homo sapiens
atggactctccggggttctc

SEQ ID NO: 116 - DNA, 20 bp, Homo sapiens
atggaaggtccggggttctc

SEQ ID NO: 117 - DNA, 20 bp, Homo sapiens
atggactctggaggggtctc

SEQ ID NO: 118 - DNA, 20 bp, Homo sapiens
atggaggctccatggggctc

SEQ ID NO: 119 - DNA, 20 bp, Homo sapiens
atggactctggggggttctc

SEQ ID NO: 120 - DNA, 20 bp, Homo sapiens
atggactctggggggttctc

SEQ ID NO: 121 - DNA, 20 bp, Homo sapiens
tccatgtgggtggggatgct

SEQ ID NO: 122 - DNA, 20 bp, Homo sapiens
tccatgcgggtggggatgct

SEQ ID NO: 123 - DNA, 20 bp, Homo sapiens
tccatgggggtcctgatgct

SEQ ID NO: 124 - DNA, 20 bp, Homo sapiens
tccatgggggtcctgatgct

SEQ ID NO: 125 - DNA, 20 bp, Homo sapiens
tccatgtggggcctgatgct

SEQ ID NO: 126 - DNA, 20 bp, Homo sapiens
tccatgtggggcctgatgct

SEQ ID NO: 127 - DNA, 20 bp, Homo sapiens
tccatggggtccctgatgct

SEQ ID NO: 128 - DNA, 20 bp, Homo sapiens
tccatggggtgcctgatgct

SEQ ID NO: 129 - DNA, 20 bp, Homo sapiens
tccatggggttcctgatgct

SEQ ID NO: 130 - DNA, 20 bp, Homo sapiens
tccatggggtccctgatgct

SEQ ID NO: 131 - DNA, 20 bp, Homo sapiens
tccatcgggggcctgatgct

SEQ ID NO: 132 - DNA, 14 bp, Homo sapiens
gctagagggagtgt

SEQ ID NO: 133 - DNA, 20 bp, Homo sapiens
gggggggggggggggggggg

SEQ ID NO: 134 - DNA, 21 bp, Homo sapiens
actgacagactgacagactga

SEQ ID NO: 135 - DNA, 21 bp, Homo sapiens
agtgacagacagacacactga

SEQ ID NO: 136 - DNA, 21 bp, Homo sapiens
actgacagactgatagaccca

SEQ ID NO: 137 - DNA, 21 bp, Homo sapiens
agtgagagactgcaagactga

SEQ ID NO: 138 - DNA, 21 bp, Homo sapiens
aatgccagtccgacaggctga

SEQ ID NO: 139 - DNA, 21 bp, Homo sapiens
ccagaacagaagcaatggatg

SEQ ID NO: 140 - DNA, 21 bp, Homo sapiens
cctgaacagaagccatggatg

SEQ ID NO: 141 - DNA, 21 bp, Homo sapiens
gcagaacagaagacatggatg

SEQ ID NO: 142 - DNA, 21 bp, Homo sapiens
ccacaacacaagcaatggata

SEQ ID NO: 143 - DNA, 21 bp, Homo sapiens
aagctagccagctagctagca

SEQ ID NO: 144 - DNA, 21 bp, Homo sapiens
cagctagccacctagctagca

SEQ ID NO: 145 - DNA, 21 bp, Homo sapiens
aagctaggcagctaactagca

SEQ ID NO: 146 - DNA, 21 bp, Homo sapiens
gagctagcaagctagctagga

SEQ ID NO: 147 - DNA, 10 bp, Homo sapiens
  misc_feature (3)..(3): n is a, c, g, or t
tcntnncgnn

SEQ ID NO: 148 - DNA, 7 bp, Homo sapiens
  misc_feature (4)..(4): n is a, c, g, or t
gggnggg

SEQ ID NO: 149 - DNA, 11 bp, Homo sapiens
  misc_feature (4)..(4): n is a, c, g, or t
gggngggnggg

SEQ ID NO: 150 - DNA, 22 bp, Homo sapiens
tcgtcgttgtcgttttgtcgtt

SEQ ID NO: 151 - DNA, 24 bp, Homo sapiens
ctggtctttctggtttttttctgg

SEQ ID NO: 152 - DNA, 27 bp, Homo sapiens
tcgcgtgcgttttgtcgttttgacgtt

SEQ ID NO: 153 - DNA, 23 bp, Homo sapiens
tcgtcgtttgtcgttttgtcgtt

SEQ ID NO: 154 - DNA, 20 bp, Homo sapiens
gggggacgatcgtcgggggg

(19) United States
(12) Patent Application Publication
(10) Pub. No.: US 2005/0238660 A1
(43) Pub. Date: Oct. 27, 2005

(54) Cpg formulations and related methods

(76) Inventors: Lorne Alan Babiuk, Saskatoon (SK); Rolf Hecker, Dusseldorf (DE)

Correspondence Address: Judy Jarecki-Black;Merial Ltd, 3239 Satellite Blvd, Duluth, GA, 30096, US

(21) Appl. No.: 10/492,002

(22) PCT Filed: Oct. 7, 2002
(86) PCT No.: PCT/EP02/11206

§ 371 Date: Apr. 6, 2004

(30) Foreign Application Priority Data
Oct. 6, 2001 (US) 60327734

(51) Int. Cl. A61K 48/00; A61K 39/00; A61K 31/7076; A61K 31/7072; A61K 31/551; A61K 39/12; A61K 31/522

(52) U.S. Cl. 424/204.1; 514/44; 514/11; 514/45; 514/49; 514/220; 514/263.31

(57) ABSTRACT

The invention involves methods and compositions of an immunostimulatory nucleic acid in combination with other therapeutic formulations such as oil-in-water emulsions. The combination of therapeutics are administered in various dosages or at various time schedules for the treatment of disorders such as disease and cancer.

### FIELD OF THE INVENTION

The present invention relates to the use of immunostimulatory nucleic acids in combination with other therapeutic formulations.

### BACKGROUND OF THE INVENTION

In United States alone the death rate due to infectious disease rose 58% between 1980 and 1992. During this time, the use of anti-infective therapies to combat infectious disease has grown significantly and is now a multi-billion dollar a year industry. Even with these increases in anti-infective agent use, the treatment and prevention of infectious disease remains a challenge to the medical community throughout the world. In general, there are three types of anti-infective agents, anti-bacterial agents, anti-viral agents, and anti-fungal agents, and even within these classes of agents there is some overlap with respect to the type of microorganism they are useful for treating.

One of the problems with anti-infective therapies is the side effects occurring in the host that is treated with the anti-infective. For instance, many anti-infectious agents can kill or inhibit abroad spectrum of microorganisms and are not specific for a particular type of species. Treatment with these types of anti-infectious agents results ill the killing of the normal microbial flora living in the host, as well as the infectious microorganism. The loss of the microbial flora can lead to disease complications and predispose the host to infection by other pathogens, since the microbial flora compete with and function as barriers to infectious pathogens. Other side effects may arise as a result of specific or non-specific effects of these chemical entities on non-microbial cells or tissues of the host.

In addition to anti-infective agents, vaccines are used to prevent and treat infectious disease. Vaccines include an antigen in combination with an adjuvant Adjuvants play an important role in the efficacy of vaccines of the treatment and prevention of infectious disease. In addition to increasing the strength and kinetics of an immune response, adjuvants also play a role in determining the type of immune response generated. Aluminum compounds, including aluminum hydroxide and aluminum phosphate, are widely used with human vaccines. These adjuvants skew the immune response towards a T-helper type 2 (Th2) response, which is characterized by the secretion of Th2 type cytokines such as IL-4 and IL-5 and the generation of IgG1 and IgE type antibodies, but weak or absent cytotoxic T lymphocyte (CTL) responses (Bomford, R 1998. Will adjuvants be needed for vaccines of the future? Dev.Biol.Stand, 92:13-17; Brazolot Millan, C. L., et al. 1998. CpG DNA can induce strong Th1 humoral aid cell-mediated immune responses against hepatitis B surface antigen in young mice. Proc.Natl.Acad.Sci. USA 95:15553-15558; Davis, M. L., et al. 1998. CpG DNA is a potent enhancer of specific immunity in mice immunized with recombinant hepatitis B surface antigen. J.Immunol 160:870-876). Development of the appropriate type of immune response is essential for successful immunization. Strong innate immunity that is associated with a Th1 type immune response, is thought to be essential for the control of intracellular pathogens, whereas strong humoral immunity, which can be found with both Th1 and Th2 type immune responses, appears to be essential for the control of extracellular pathogens (Constant, S. L. and K. Bottomly. 1997. Induction of Th1 and Th2 CD4+T cell responses: the alternative approaches. Ann.Rev.Immunol. 15:297-322). Synthetic oligodeoxynucleotides containing unmethylated CpG dinucleotides (CpG ODN) are novel adjuvants known to promote Th1 type immune responses with the secretion of IFN-γ, TNF-α and IL12 cytokines, opsonizing antibodies such as those of the IgG2a isotype, and strong CTL induction (Chu, R. S., et al. 1997. CpG oligodeoxynucleotides act as adjuvants that switch on T helper 1 (Th1)immunity. J.Exp.Med 186:1623-1631; Klinman, D. M. et al. 1999. CpG motifs as immune adjuvants. Vaccine 17:19-25).

Bovine herpesvirus-1 (BHV-1), a member of the alphaherpesvirinae subfamily, is associated with a variety of clinical disease manifestations including rhinotracheitis, vulvovaginitis, abortions, conjunctivitis, encephalitis and generalized systemic infections (Gibbs, E. P. J. and M. M. Rweyemamu. 1977. Bovine herpesvirus-1, p. 317 Anonymous Bovine herpesvirus. Vet. Bull, London; Yates, W. D. G. 1982. A review of infectious bovine rhinotracheitis, shipping fever pneumonia and viral-bacterial synergism in respiratory disease of cattle. Can.J.Comp.Med. 46:225-263). Bovine respiratory diseases cost the cattle industry up to $1 billion per year in North America (Yates, W. D. G. 1982. A review of infectious bovine rhinotracheitis, shipping fever pneumonia and viral-bacterial synergism in respiratory disease of cattle. Can.J.Comp.Med. 46:225-263). These losses occur even though live attenuated and killed vaccines are available. At present, the greatest potential for combined efficacy, safety, antigenic specificity and protection against BHV-1 resides in subunit vaccines consisting of one or more of the viral glycoproteins, gB, gC and gD and an adjuvant Conventional adjuvants such as VSA3, however, not only generate a Th2-like immune response, but are not metabolized and leave injection site reactions. Such reactions are unacceptable for human or veterinary vaccines.

### SUMMARY OF THE INVENTION

The invention provides improved methods and products for the treatment of subjects using immunostimulatory nucleic acids in combination with specific formulations. The invention is based, in part, on the finding that when some types of immunostimulatory nucleic acid molecules are used in conjunction with specific formulations, some unexpected and improved results are observed For instance, the efficacy of the combination of some lo immunostimulatory nucleic acids and the formulation is profoundly improved over the use of the immunostimulatory nucleic acid alone. The results are surprising, in part, because the immunostimulatory nucleic acids and the formulations act through different mechanisms and would not necessarily be expected to improve the efficacy of the other in a synergistic manner.

In one aspect the invention relates to a method for reducing viral shedding in a non-human animal by administering to a non-human animal infected with a virus or at risk of viral infection, an immunostimulatory nucleic acid and an oil-in-water emulsion in an effective amount to reduce viral shedding. In one embodiment the oil-in-water emulsion is EMULSIGEN™. Optionally the non-human animal is a dog, cat, horse, cow, pig, sheep, goat, primate or chicken.

The combination of active agents may be administered with or without an antigen or an antiviral agent. In some embodiments the antiviral agent is selected from the group consisting of Acemannan; Acyclovir, Acyclovir Sodium; Adefovir; Alovudine; Alvircept Sudotox; Amantadine Hydrochloride; Aranotin; Arildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxime; Famciclovir; Famotine Hydrochloride; Fiacitabine; Fialuridine; Fosarilate; Foscarnet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadane Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

In other aspects the invention is a method for reducing tissue damage upon vaccination of a subject by administering to a subject by an invasive route an adjuvanted vaccine and an immunostimulatory nucleic acid in an effective amount to reduce tissue damage arising from the adjuvanted vaccine, wherein the vaccine is adjuvanted with an oil-in-water emulsion. In one embodiment the oil-in-water emulsion is EMULSIGEN™. The invasive route may be any type of route that produces an opening in a tissue barrier, such as skin. In some embodiments the invasive route is subcutaneous or intramuscular.

According to other aspects the invention is a method for inducing an immune response by administering to a subject an oil-in-water emulsion and a CpG oligonucleotide in an effective amount to produce the immune response. Optionally the immune response is an antigen specific immune response and the subject is administered an antigen. In one embodiment the oil-in-water emulsion is EMULSIGEN™.

According to yet other aspects the invention relates to a method for reducing a dosage of antigen adiministered to a subject to produce an antigen specific immune response by administering to a subject an antigen in a sub-therapeutic dosage and an immunostimulatory nucleic acid, wherein the combination of the sub-therapeutic dose of the antigen and the immunostimulatory nucleic acid produce an antigen specific immune response. In some embodiments the sub-therapeutic dose of the antigen is a dose which is at least 50% less than a minimal effective dose of antigen for producing an antigen specific immune response when the antigen is formulated with alum. Alternatively the sub-therapeutic dose of the antigen may be a dose which is at least 90% less than a minimal effective dose of antigen for producing an antigen specific immune response when the antigen is formulated with alum.

The methods of the invention involve the use of an immunostimulatory nucleic acid. The immunostimulatory nucleic acid may be a CpG oligonucleotide and in some embodiments is 2007 (TCGTCGTTGTCGTMTGTCGTT); 2142 (TCGCGTGCGTTTTGTCGTTTTGACGTT); 2135 (TCGTCGTTTGTTGTCGTTTTGTCGGTT); and/or 2216 (ggGGGACGATCGTCgggggG). Alternatively the immunostimulatory nucleic acid may be a T-rich nucleic acid, such as the ODN of SEQ ID NO:52 -57 and/or SEQ ID NO: 62-94 or a poly-G nucleic acid such as the ODN of SEQ ID NO: 46, SEQ D NO: 47, SEQ ID NO: 58, SEQ ID-NO: 61, and/or SEQ ID NO: 95-133. In other embodiments the immunostimulatory nucleic acid may have a sequence selected from the group consisting of SEQ ID NO: 1 through to SEQ ID NO: 146.

The immunostimulatory nucleic acid, such as the CpG oligonucleotide may be administered a single time or multiple times. If the CpG oligonucleotide is administered multiple times it may be administered at regular intervals, such as, for example, on a weekly basis, on a daily basis, or on a monthly basis.

The immunostimulatory nucleic acid, such as the CpG oligonucleotide may be administered by any route. For instance the immunostimulatory nucleic acid may be administered orally, by injection, or through a sustained release device.

In some embodiments of the invention the subject has a cancer or an infectious disease. In other embodiments the subject is at risk of developing a cancer or an infectious disease. Optionally the subject has a cancer selected from the group consisting of bone cancer, brain and CNS cancer, connective tissue cancer, esophageal cancer, eye cancer, Hodgkin's lymphoma, larynx cancer, oral cavity cancer, skin cancer, and testicular cancer. The subject may also be an immunocompromised subject. In other embodiments the subject has an infectious disease selected from the group consisting of a viral bacterial, fungal and parasitic infection In yet another embodiment the subject is at risk of developing an infectious disease elected from the group consisting of a viral, bacterial, fungal and parasitic infection.

The immunostimulatory nucleic acid may have a modified backbone, such as a phosphate modified backbone or a peptide modified oligonucleotide backbone. In one embodiment the phosphate modified backbone is a phosphorothioate modified backbone.

In other aspects the invention is a composition of an immunostimulatory nucleic acid and an oil-in-water emulsion. In one embodiment the oil-in-water emulsion is EMULSIGEN™.

In certain embodiments of all aspects of the invention, the immunostimulatory nucleic acid may be a nucleic acid which stimulates a Th1 immune response. Similarly, in some aspects of the invention, it is conceivable that one or more different immunostimulatory nucleic acids may be administered to a subject. Thus depending on the embodiment, one, two, three, four, five or more different immunostimulatory-nucleic acids may be administered to a subject in a particular method. Thus, the term “an immunostimulatory nucleic acid” is meant to embrace a single immunostimulatory nucleic acid, a plurality of immunostimulatory nucleic acids of a particular class and a plurality of immunostimulatory nucleic acids of different classes.

According to other embodiments, the immunostimulatory nucleic acid is administered concurrently with, prior to, or following the administration of the other therapeutic formulation, e.g., oil-in-water emulsion, antigen etc.

In some embodiments, the immunostimulatory nucleic acid is administered in an effective amount for upregulating, enhancing or activating an immune response. In some embodiments, the immunostimulatory nucleic acid is administered in an effective amount for redirecting the immune response from a Th2 to a Th1 immune response. In still other embodiments, a plurality of immunostimulatory nucleic acids, with different nucleic acid sequences and with different functional effects, is administered.

Each of the limitations of the invention can encompass various embodiments of the invention. It is, therefore, anticipated that each of the limitations of the invention involving any one element or combinations of elements can be included in each aspect of the invention

### BRIEF DESCRIPTION OF THE FIGURES

FIG. 1 is a bar graph depicting BHV-1 neutralizing antibody responses in the serum of vaccinated and control animals 14, 47 and 64 days after primary immunization (Imm 1 (day 14); 14 days after primary immunization, Imm 2 (day 47); 8 days after secondary immunization, post chall (day 64); 11 days after viral challenge). Antibody titers are expressed as a 50% endpoint using 100 PFU of BHV-1. Error bars show the standard error of the geometric means of seven animals.

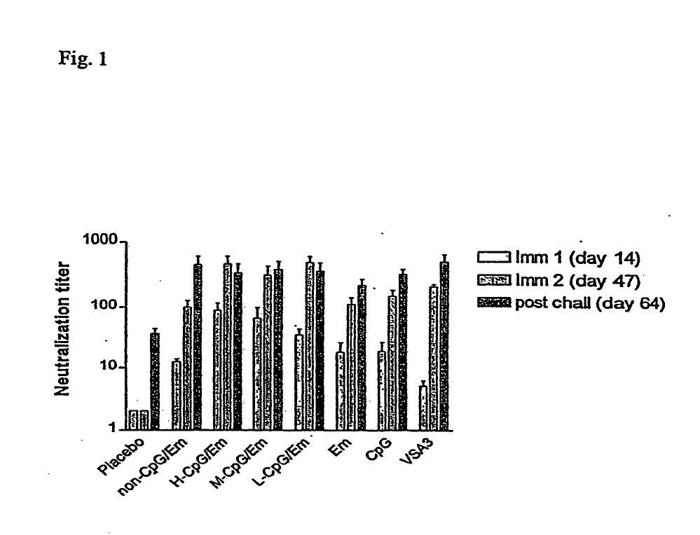

FIG. 2 is three bar graphs depicting cellular immune responses after vaccination. Data are expressed as average±standard error of the mean. (a) Antigen-specific proliferation of PBMC before and after challenge. Stimulated index represents the counts per min in the presence of antigen divided by counts per min in the absence of antigen. (b) Difference in the number of spots per 10⁶ cells in antigen-stimulated wells and the number of spots per 10⁶ cells in nonstimulated wells. (c) Amount of IFN-γ secreted by PBMC in response to BHV-1 gD after 24 hours.

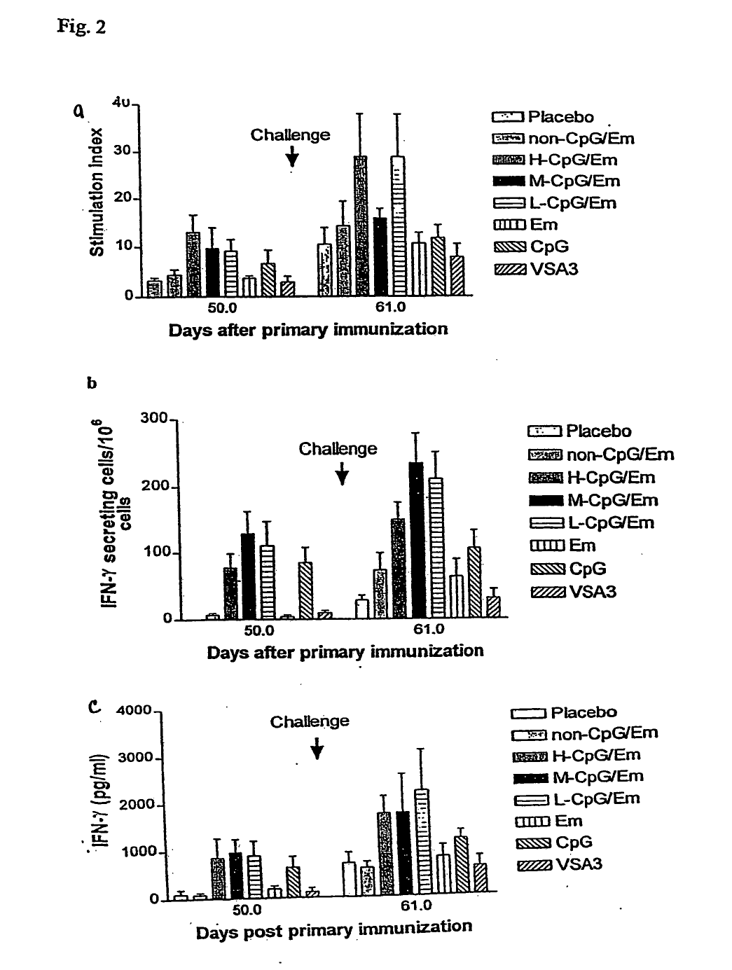

FIG. 3 is two bar graphs depicting serum antibodies against BHV-1 glycoproteins 8 days after secondary immunization (Imm 2) and 11 days viral infection (after challenge). (a) Antibodies against tgD (b) Antibodies against tgB.

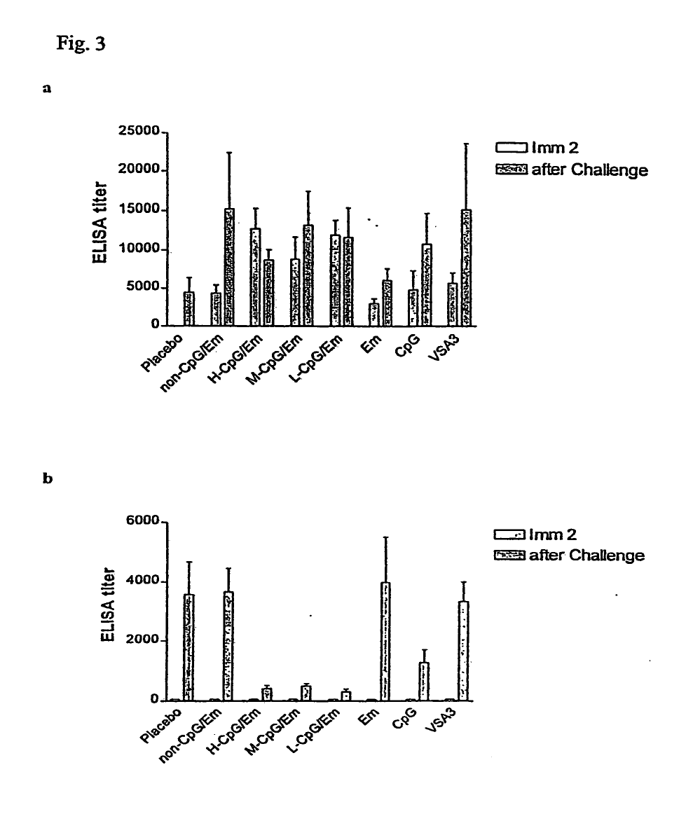

FIG. 4 is two graphs depicting the effect of immunization on rectal temperature in animals challenged with BHV-1. a. mean temperature response b. number of fever days; total number of days temperature was ≧40° C.

FIG. 5 is two graphs depicting the effect of immunization on weight gain in animals challenged with BHV-1. (a) Cumulative weight change. (b) Number of animal days weight loss was above of below 5 kg.

FIG. 6 is a graph depicting the extent of viral replication following −1 challenge. On the day of challenge and on alternative days thereafter, virus titres were determined in the nasal secretions of immunized animals. Error bars show the standard error of the geometric means of seven animals.

### DETAILED DESCRIPTION OF THE INVENTION

It was surprisingly discovered according to the invention that select combinations of immunostimulatory nucleic acids and therapeutic formulations such as oil-in-water emulsions work dramatically better, and sometimes even synergistically, to improve an immune response than either component alone. Although many formulations have been developed and tested for administering drugs, these particular types dramatically enhance the activity of the immunostimulatory nucleic acids. This was surprising, in part, because other similar formulations did not demonstrate the same dramatic types of improvements as the therapeutic formulations described herein. The term “therapeutic formulations” as used herein refers to oil-in-water emulsions such as EMULSIGEN™.

As demonstrated in the Examples described below the combination of immunostimulatory nucleic acids has demonstrated significantly improved therapeutic effects in the treatment and prevention of infectious disease. Recently, it was shown that the combination of CpG ODN with alum had great potential to augment immune responses in mice with minimal side effects at the injection site compared with other adjuvant combinations (Weeratna, R. D., et al. 2000. CpG DNA induces stronger immune responses with less toxicity than other adjuvants. Vaccine 18:1755-1762). However, despite the promising results in mice, it was found that BHV-1 tgD adjuvanted with a combination of CpG ODN and alum induced similar immune responses to tgD adjuvanted with CpG ODN alone, and failed to completely protect calves from BHV-1 challenge. In addition, BHV-1 subunit vaccines adjuvanted with Freund's incomplete adjuvant also failed to protect calves from BHV-1 challenge (Israel, B. A., et al. 1988. Epitope specificity and protective efficacy of the bovine immune response to bovine herpesvirus-1 glycoprotein vaccines. Vaccine 6:349-356).

Surprisingly it has been discovered according to the invention that a BHV-1 tgD vaccine co-adjuvanted with CpG ODN and an oil-in-water emulsion, such as EMULSIGEN™ induced a stronger and more balanced immune response as well as provided a greater protection from BHV-1 challenge than tgD adjuvanted with CpG ODN, VSA3 or EMULSIGEN™ alone, or co-adjuvanted with a non-CpG ODN and EMULSIGEN™. Furthermore, the immune responses induced by tgD formulated with CpG ODN in the presence or absence of EMULSIGEN™ were more Th1-biased in contrast to those formulated with EMULSIGEN™, VSA3 or non-CpG ODN and EMULSIGEN™. The data demonstrates that immunization of animals with a vaccine such as BHV-1 subunit vaccines adjuvanted with CpG ODN and EMULSIGEN™, induces stronger more balanced humoral and cellular responses and a greater protection against viral infection than do vaccines adjuvanted with non-CpG ODN with EMULSIGEN™, EMULSIGEN™, CpG ODN or VSA3 alone.

In cattle, protection against BHV-1 is largely mediated by marked humoral immune responses. Indeed, strong cellular responses in the absence of high antibody titers do not fully protect against infection (Loehr, B. L., et at 2000. Gene gun-mediated DNA immunization primes development of mucosal immunity against bovine herpesvirus 1 in cattle. J. Virol. 74:6077-6086). Previous studies indicate that significant protection from BHV-1-induced disease can be achieved by subunit vaccines containing one or more of the viral glycoproteins (Gao, Y., et al. 1994. Truncated bovine herpesvirus-1 glycoprotein I (gpI) initiates a protective local immune response in its natural host. Vaccine 12:145-152; Baca-Estrada, M. E., et al. 1996. Immunogenicity of bovine herpesvirus 1 glycoprotein D in mice: effect of antigen form on the induction of cellular and humoral immune responses. Viral Immunol 9:11-22; Babiuk, L. A., L. et al. 1996. Immunology of bovine herpesvirus 1 infection. Vet.Microbiol. 53:31-42; Zhu, X., S. et al. 1997. Yeast-secreted bovine herpesvirus type 1 glycoprotein D has authentic conformational structure and immunogenicity. Vaccine 15:679-688; van Drunen Littel-vain den Hurk, S., et al. 1994. A subunit gIV vaccine, produced by transfected mammalian cells in culture, induces mucosal immunity against bovine herpesvirus-1 in cattle. Vaccine 12:1295-1302). However, due to inefficient humoral immune responses, some BHV-1 subunit vaccines induce little or no protection from challenge (Israel, B. A., et al. 1988. Epitope specificity and protective efficacy of the bovine immune response to bovine herpesvirus-1 glycoprotein vaccines. Vaccine 6:349-356). Conventional adjuvants such as VSA3 generate strong immune responses but they leave undesirable injection site reactions. VSA3 consists of a mineral oil-based emulsion and an inflammatory compound: dimethyl dioctadecyl ammonium bromide (DDA). In humans, DDA is known to induce a host of inflammatory reactions, including swelling and pain and delayed-type hypersensitivity at the site of injection (Vogel, F. R. and M. F. Powell. 1995. A compendium of vaccine adjuvants and excipients, p. 141-228. In M. F. Powell, M. J. Newman, and J. R. Burdman (eds.), Vaccine Design: the subunit and adjuvant approach. Plenum Press, New York). Because of its inflammatory tendencies, DDA is also used to induce experimental arthritis in rats (Mia, M. Y., Let al. 2000. Dimethyl dioctadecyl ammonium bromide (DDA)-induced arthritis in rats: a model of experimental arthritis. J. Autoimmun. 14:303-310).

Additionally it was discovered that enhanced protective immune responses are induced using the subcutaneous (s.c.) route of delivery. Subcutaneous administration is a useful mode of delivery in both veterinary and human practice because of its ease of administration and the immunocompetence of the skin. Generally vaccines have been administered intramuscularly (i.m.). The compositions of the invention may have even more enhanced effects when delivered s.c. than i.m.

The data presented below demonstrated that the combination of immunostimulatory nucleic acids with the therapeutic formulations resulted in a dramatic decrease in viral shedding. In fact some animals demonstrated zero viral shedding. This is an extremely important parameter because it reflects the amount of protection from infection. “Viral shedding” refers to production of viral particles at a mucosal surface by an animal infected with a virus. The presence or absence of viral shedding can be determined by taking a sample from an animal (i.e., nasal secretions) and analyzing the sample for the presence of virus. If a drug prevents viral shedding it effectively prevents infection in the animal. The ability of the nucleic acids in the therapeutic formulations of the invention to reduce and even eliminate viral shedding demonstrates the surprising potency of the composition.

Thus the immunostimulatory nucleic acids combined with the therapeutic formulations stimulate the immune system to prevent or treat infectious disease. The strong yet balanced, cellular and humoral immune responses that result from the immune stimulatory capacity of the nucleic acid reflect the natural defense system of the subject against invading microorganisms.

As used herein, the term “prevent”, “prevented”, or “preventing” and “treat”, “treated” or “treating” when used with respect to the prevention or treatment of an infectious disease refers to a prophylactic treatment which increases the resistance of a subject to a microorganism or, in other words, decreases the likelihood that the subject will develop an infectious disease to the microorganism, as well as to a treatment after the subject has been infected in order to fight the infectious disease, e.g., reduce or eliminate it altogether or prevent it from becoming worse.

The immunostimulatory nucleic acids are useful for treating or preventing infectious disease in a subject. A “subject” shall mean a human or vertebrate mammal including but not limited to a dog, cat, horse, cow, pig, sheep, goat, or primate, e.g., monkey. In some embodiments a subject specifically excludes rodents such as mice.

The immunostimulatory nucleic acids are useful in some aspects of the invention as a prophylactic for the treatment of a subject at risk of developing an infectious disease where the exposure of the subject to a microorganism or expected exposure to a microorganism is known or suspected. A “subject at risk” of developing an infectious disease as used herein is a subject who has any risk of exposure to a microorganism, e.g. someone who is in contact with an infected subject or who is travelling to a place where a particular microorganism is found For instance, a subject at risk may be a subject who is planning to travel to an area where a particular microorganism is found or it may even be any subject living in an area where a microorganism has been identified. A subject at risk of developing an infectious disease includes those subjects that have a general risk of exposure to a microorganism, e.g., influenza, but that don't have the active disease during the treatment of the invention as well as subjects that are considered to be at specific risk of developing an infectious disease because of medical or environmental factors, that expose them to a particular microorganism.

In addition to the use of the immunostimulatory nucleic acid and the anti-microbial agent for prophylactic treatment, the invention also encompasses the use of the combination of drugs for the treatment of a subject having an infectious disease. A “subject having an infectious disease” is a subject that has had contact with a microorganism. Thus the microorganism has invaded the body of the subject The word “invade” as used herein refers to contact by the microorganism with the external surface of the subject, e.g., skin or mucosal membranes and/or refers to the penetration of the external surface of the subject by the microorganism.

An “infectious disease” as used herein, refers to a disorder arising from the invasion of a host, superficially, locally, or systemically, by an infectious microorganism. Infectious microorganisms include bacteria, viruses, and fungi. Bacteria are unicellular organisms which multiply asexually by binary fission. They are classified and named based on their morphology, staining reactions, nutrition and metabolic requirements, antigenic structure, chemical composition, and genetic homology. Bacteria can be classified into three groups based on their morphological forms, spherical (coccus), straight-rod (bacillus) and curved or spiral rod (vibrio, campylobacter, spirillum, and spirochaete). Bacteria are also more commonly characterized based on their staining reactions into two classes of organisms, gram-positive and gram-negative. Gram refers to the method of staining which is commonly performed in microbiology labs. Gram-positive organisms retain the stain following the staining procedure and appear a deep violet color. Gram-negative organisms do not retain the stain but take up the counter-stain and thus appear pink.

Infectious bacteria include, but are not limited to, gram negative and gram positive bacteria. Gram positive bacteria include, but are not limited to Pasteurella species, Staphylococci species, and Streptococcus species. Gram negative bacteria include, but are not limited to, Escherichia coli, Pseudomonas species, and Salmonella species. Specific examples of infectious bacteria include but are not limited to: Helicobacter pyloris, Borelia burgdorferi, Legionella pneumophilia, Mycobacteria sps (e.g. M. tuberculosis, M. avium, M. Intracellular, M. kansaii, M. gordonae), Staphylococcus aureus, Neisseria gonorrhoeae, Neisseria meningitidis, Listeria monocytogenes, Streptococcus pyogenes (Group A Streptococcus), Streptococcus agalactiae (Group B Streptococcus), Streptococcus (viridans group), Streptococcus faecalis, Streptococcus bovis, Streptococcus (anaerobic species.), Streptococcus pneumoniae, pathogenic Campylobacter sp., Enterococcus sp., Haemophilus influenzae, Bacillus antracis, corynebacterium diphtheriae, corynebacterium sp., Erysipelothrix rhusiopathiae, Clostridium perfringens, Clostridium tetani, Enterobacter aerogenes, Klebsiella pneumoniae, Pasturella multocida, Bacteroides sp., Fusobacterium nucleatum, Streptobacillus moniliformis, Treponema pallidium, Treponema pertenue, Leptospira, Rickettsia, and Actinomyces Israeli.

Viruses are small infectious agents which contain a nucleic acid core and a protein coat, but are not independently living organisms. A virus cannot survive in the absence of a living cell within which it can replicate. Viruses enter specific living cells either by endocytosis or direct injection of DNA (phage) and multiply, causing disease. The multiplied virus can then be released and infect additional cells. Some viruses are DNA-containing viruses and other are RNA-containing viruses.

Once the virus enters the cell it can cause a variety of physiological effects. One effect is cell degeneration, in which the accumulation of virus within the cell causes the cell to die and break into pieces and release the virus. Another effect is cell fusion, in which infected cells fuse with neighboring cells to produce syncytia. Other types of virus cause cell proliferation which results in tumor formation.

Viruses include, but are not limited to, interoviruses (including, but not limited to, viruses that the family picornaviridae, such as polio virus, coxsackie virus, echo virus), rotaviruses, adenovirus, hepatitus. Specific examples of viruses that have been found in humans include but are not limited to: Retroviridae (e.g. human immunodeficiency viruses, such as HIV-1 (also referred to as HTLV-III, LAV or HTLV-III/LAV, or HIV-III; and other isolates, such as HIV-LP; Picornaviridae (e.g. polio viruses, hepatitis A virus; enteroviruses, human Coxsackie viruses, rhinoviruses, echovirses); Calciviridae (e.g. strains that cause gastroenteritis); Togaviridae (e.g. equine encephalitis viruses, rubella viruses); Flaviridae (e.g. dengue viruses, encephalitis viruses, yellow fever viruses); Coronoviridae (e.g. coronaviruses); Rhabdoviradae (e.g. vesicular stomatitis viruses, rabies viruses); Coronaviridae (e.g. coronaviruses); Rhabdoviradae (e.g. vesicular stomatitis viruses, rabies viruses); Filoviridae (e.g. ebola viruses); Paramyxoviridae (e.g. parainfluenza viruses, mumps virus, measles virus, respiratory syncytial virus); Orthomyxoviridae (e.g. influenza viruses); Bungaviridae (e.g. Hantaan viruses, bunga viruses, phleboviruses and Nairo viruses); Arena viridae (hemorrhagic fever viruses); Reoviridae (e.g. reoviruses, oxbiviurses and rotaviruses); Birnaviridae; Hepadnaviridae (Hepatitis B virus); Parvovirida (parvoviruses); Papovaviridae (papilloma viruses, polyoma viruses); Adenoviridae (most adenoviruses); Herpesviridae (herpes simplex virus USV) 1 and 2, varicella zoster virus, cytomegalovirus (CMV), herpes virus; Poxviridae (variola viruses, vaccinia viruses, pox viruses); and Iridoviridae (e.g. African swine fever virus); and unclassified viruses (e.g. the etiological agents of Spongiform encephalopathies, the agent of delta hepatitis (thought to be a defective satellite of hepatitis B virus), the agents of non-A, non-B hepatitis (class 1=inteally transmitted; class 2=parenterally transmitted (i.e. Hepatitis C); Norwalk and related viruses, and astroviruses).

In addition to viruses that infect human subjects causing human disorders, the invention is also useful for treating other non-human vertebrates. Non-human vertebrates are also capable of developing infections which can be prevented or treated with the combinations of immunostimulatory nucleic acids and anti-microbials disclosed herein. For instance, in addition to the treatment of infectious human diseases, the methods of the invention are useful for treating or preventing infections of non-human animals.

Infectious virus of both human and non-human vertebrates, include retroviruses, RNA viruses and DNA viruses. This group of retroviruses includes both simple retroviruses and complex retroviruses. The simple retroviruses include the subgroups of B-type retoviruses, C-type retroviruses and D-type retroviruses. An example of a B-type retrovirus is mouse mammary tumor virus (MMTV). The C-type retroviruses include subgroups C-type group A (including Rous sarcoma virus (RSV), avian leukemia virus (ALV), and avian myeloblastosis virus (AMV)) and C-type group B (including murine leukemia virus (MLV), feline leukemia virus (FeLV), murine sarcoma virus (MSV), gibbon ape leukemia virus (GALV), spleen necrosis virus (SNV), reticuloendotheliosis virus (RV) and simian sarcoma virus (SSV)). The D-type retroviruses include Mason-Pfizer monkey virus (MPMV) and simian retrovirus type 1 (SRV-1). The complex retroviruses include the subgroups of lentiviruses, Tell leukemia viruses and the foamy viruses. Lentiviruses include HIV-1, but also include HV-2, SIV, Visna virus, feline immunodeficiency virus (FIV), and equine infectious anemia virus (EIAV). The T-cell leukemia viruses include HTLV-1, HTLV-II simian T-cell leukemia virus (STLV), and bovine leukemia virus (BLV). The foamy viruses include human foamy virus (HFV), simian foamy virus (SFV) and bovine foamy virus (BFV).

Examples of other RNA viruses that are antigens in vertebrate animals include, but are not limited to, the following: members of the family Reoviridae, including the genus Orthoreovirus (multiple serotypes of both mammalian and avian retroviruses), the genus Orbivirus (Bluetongue virus, Eugenangee virus, Kemerovo virus, African horse sickness virus, and Colorado Tick Fever virus), the genus Rotavirus (human rotavirus, Nebraska calf diarrhea virus, murine rotavirus, simian rotavirus, bovine or ovine rotavirus, avian rotavirus); the family Picornaviridae, including the genus Enterovirus (poliovirus, Coxsackie virus A and B, enteric cytopathic human orphan (ECHO) viruses, hepatitis A virus, Simian enteroviruses, Murine encephalomyelitis (ME) viruses, Poliovirus muris, Bovine enteroviruses, Porcine enteroviruses, the genus Cardiovirus (Encephalomyocarditis virus (EMC), Mengovirus), the genus Rhinovirus (Human rhinoviruses including at least 113 subtypes; other rhinoviruses), the genus Apthovirus (Foot and Mouth disease (FMDV); the family Calciviridae, including Vesicular exanthema of swine virus, San Miguel sea lion virus, Feline picornavirus and Norwalk virus; the family Togaviridae, including the genus Alphavirus (Eastern equine encephalitis virus, Semliki forest virus, Sindbis virus, Chikungunya virus, O'Nyong-Nyong virus, Ross river virus, Venezuelan equine encephalitis virus, Westem equine encephalitis virus), the genus Flavirius (Mosquito borne yellow fever virus, Dengue virus, Japanese encephalitis virus, St. Louis encephalitis virus, Murray Valley encphalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus (Bunyamwera and related viruses, California encephalitis group virses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukmiemi and related viruses); the family Orthomyxoviridae, including the genus Influenza virus (Influenza virus type A, many human subtypes); Swine influenza virus, and Avian and Equine Influenza viruses; influenza type B (many human subtypes), and influenza type C (possible separate genus); the family paramyxoviridae, including the genus Paramyxovirus (Parainfuenza virus type 1, Sendai virus, Hemadsorption virus, Parainfluenza viruses types 2 to 5, Newcastle Disease Virus, Mumps virus), the genus Morbillivirus Measles virus, subacute sclerosing panencephalitis virus, distemper virus, Rinderpest virus), the genus Pneumovirus (respiratory syncytial virus (RSV), Bovine respiratory syncytial virus and Pneumonia virus of mice); forest virus, Sindbis virus, Chikungunya virus, O'Nyong-Nyong virus, Ross river virus, Venezuelan equine encephalitis virus, Western equine encephalitis virus), the genus Flavirius (Mosquito bome yellow fever virus, Dengue virus, Japanese encephalitis virus, St. Louis encephalitis virus, Murray Valley encephalitis virus, West Nile virus, Kunjin virus, Central European tick borne virus, Far Eastern tick borne virus, Kyasanur forest virus, Louping III virus, Powassan virus, Omsk hemorrhagic fever virus), the genus Rubivirus (Rubella virus), the genus Pestivirus (Mucosal disease virus, Hog cholera virus, Border disease virus); the family Bunyaviridae, including the genus Bunyvirus Bunyamwera and related viruses, California encephalitis group viruses), the genus Phlebovirus (Sandfly fever Sicilian virus, Rift Valley fever virus), the genus Nairovirus (Crimean-Congo hemorrhagic fever virus, Nairobi sheep disease virus), and the genus Uukuvirus (Uukuieni and related viruses); the family Orthomyxoviridae, including the genus Influenza virus (Influnza virus type A, many human subtypes); Swine influenza virus, and Avian and Equine Influenza virus; influenza type B (many human subtypes), and influenza type C (possible separate genus); the family paramyxoviridae, including the genus Paramyxovirus (Parainfluenza virus type 1, Sendai virus, Hemadsorption virus, Parainfluenza viruses types 2 to 5, Newcastle-Disease Virus, Munps virus), the genus Morbillivirus (Measles virus, subacute sclerosing panencephalitis virus, distemper virus, Rinderpest virus), the genus Pneumovirus (respiratory syncytia virus (RSV), Bovine respiratory syncytial virus and Pneumonia virus of mice); the family Rhabdoviradae, including the genus Vesiculovirus (VSV), Chandipura virus, Flanders-Hart Park virus), the genus Lyssavirus (Rabies virus), fish Rhabdoviruses, and two probable Rhabdoviruses (Marburg virus and Ebola virus); the family Arenaviridae, including Lymphocytic choriomeningitis virus (LCM), Tacaribe virus complex, and Lassa virus; the family Coronoaviridae, including Infectious Bronchitis Virus (IBV), Mouse Hepatitis virus, Human enteric corona virus, and Feline infectious peritonitis (Feline coronavirus).

Illustrative DNA viruses that infect vertebrate animals include, but are not limited to: the family Poxviridae, including the genus Orthopoxvirus (Variola major, Variola minor, Monkey pox Vaccinia, Cowpox, Buffalopox, Rabbitpox, Ectromelia), the genus Leporipoxvirus Myxoma, Fibroma), the genus Avipoxvirus (Fowlpox, other avian poxvirus), the genus Capripoxvirus (sheeppox, goatpox), the. genus Suipoxvirus (Swinepox), the genus Parapoxvirus (contagious postular dermatitis virus, pseudocowpox, bovine papular stomatitis virus); the family Iridoviridae (African swine fever virus, Frog viruses 2 and 3, Lymphocystis virus of fish); the family Herpesviridae, including the alpha-Herpesviruses (Herpes Simplex Types 1. and 2, Varicella-Zoster, Equine abortion virus, Equine herpes virus 2 and 3, pseudorabies virus, infectious bovine keratoconjunctivitis virus, infectious bovine rhinotracheitis virus, feline rhiniotracheitis virus, infectious laryngotracheitis virus) the Beta-herpesviruses Human cytomegalovirus and cytomegaloviruses of swine, monkeys and rodents); the gamma-herpesviruses (Epstein-Barr virus (EBV), Marek's disease virus, Herpes saimiri, Herpesvirus ateles, Herpesvirus sylvilagus, guinea pig herpes virus, Lucke tumor virus); the family Adenoviridae, including the genus Mastadenovirus (Hurman subgroups A,B,C,D,E and ungrouped; san adenoviruses (at least 23 serotypes), infectious canine hepatitis, and adenoviruses of cattle, pigs, sheep, frogs and many other species, the genus Aviadenovirus (Avian adenoviruses); and non-cultivatable adenoviruses; the family Papoviridae, including the genus Papillomavirus (Human papilloma viruses, bovine papilloma viruses, Shope rabbit papilloma virus, and various pathogenic papilloma viruses of other species), the genus Polyomavirus (polyomavirus, Simian vacuolating agent (SV-40), Rabbit vacuolating agent (RKV), K virus, BK virus, JC virus, and other primate polyoma viruses such as Lymphotrophic papilloma virus); the family Parvoviridae including the genus Adeno-associated viruses, the genus Parvovirus (Feline panleukopenia virus, bovine parvovirus, canine parvovinis, Aleutian mink disease virus, etc). Finally, DNA viruses may include viruses which do not fit into the above families such as Kuru and Creutzfeldt-Jacob disease viruses and chronic infectious neuropathic agents (CHINA virus).

Fungi are eukaryotic organisms, only a few of which cause infection in vertebrate mammals. Because fungi are eukaryotic organisms, they differ significantly from prokaryotic bacteria in size, structural organization, life cycle and mechanism of multiplication. Fungi are classified generally based on morphological features, modes of reproduction and culture characteristics. Although fungi can cause different types of disease in subjects, such as respiratory allergies following inhalation of fungal antigens, fungal intoxication due to ingestion of toxic substances, such as amatatoxin and phallotoxin produced by poisonous mushrooms and aflotoxins, produced by aspergillus species, not all fungi cause infectious disease.

Infectious fungi can cause systemic or superficial infections. Primary systemic infection can occur in normal healthy subjects and opportunistic infections, are most frequently found in immuno-compromised subjects. The most common fungal agents causing primary systemic infection include blastomyces, coccidioides, and histoplasma. Common fungi causing opportunistic infection in immuno-compromised or immunosuppressed subjects include, but are not limited to, candida albicans (an organism which is normally part of the respiratory tract flora), cryptococcus neoformans (sometimes in normal flora of respiratory tract), and various aspergillus species. Systemic fungal infections are invasive infections of the internal organs. The organism usually enters the body through the lungs, gastrointestinal tract, or intravenous lines. These types of infections can be caused by primary pathogenic fungi or opportunistic fungi.

Superficial fungal infections involve growth of fungi on an external surface without invasion of internal tissues. Typical superficial fungal infections include cutaneous fungal infections involving skin, hair, or nails. An example of a cutaneous infection is Tinea infections, such as ringworm, caused by dermatophytes, such as microsporum or traicophyton species, i.e., microsporum canis, microsporum gypsum, tricofitin rubrum. Examples of fungi include: Cryptococcus neoformans, Histoplasma capsulatum, Coccidioides immitis, Blastomyces dermatitidis, Chlamydia trachomatis, Candida albicans.

Parasitic infections targeted by the methods of the invention include those caused by the following parasites Plasmodium falciparum, Plasmodium ovale, Plasmodium malariae, Plasmdodium vivax, Plasmodium knowlesi, Babesia microti, Babesia divergens, Trypanosoma cruzi, Toxoplasma gondii, Trichinella spiralis, Leishmania major, Leishmania donovani, Leishmania braziliensis and Leishmania tropica, Trypanosoma gambiense, Trypanosmoma rhodesiense and Schistosoma mansoni. In preferred embodiments, the method is directed towards the prevention of infection with parasites which cause malaria.

Other medically relevant microorganisms have been described extensively in the literature, e.g., see C. G. A Thomas, Medical Microbiology, Bailliere Tindal, Great Britain 1983, the entire contents of which is hereby incorporated by reference. Each of the foregoing lists is illustrative, and is not intended to be limiting.

The methods of the invention involve combinations of immunostimulatory nucleic acids and therapeutic formulations. The combination of active agents may also be administered in conjunction with an anti-microbial agent for the treatment or prevention of infectious disease. An anti-microbial agent, as used herein, refers to a naturally-occuing or synthetic compound which is capable of killing or inhibiting infectious microorganisms. The type of anti-microbial agent useful according to the invention will depend upon the type of microorganismn with which the subject is infected or at risk of becoming infected. One type of anti-microbial agent is an antibacterial agent. Antibacterial agents kill or inhibit the growth or function of bacteria A large class of antibacterial agents is antibiotics.

Antiviral agents. are compounds which prevent infection of cells by viruses or replication of the virus within the cell. There are many fewer antiviral drugs than antibacterial drugs because the process of viral replication is so closely related to DNA replication within the host cell, that non-specific antiviral agents would often be toxic to the host. There are several stages within the-process of viral infection which can be blocked or inhibited by antiviral agents. These stages include, attachment of the virus to the host cell (immunoglobulin or binding peptides), uncoating of the virus (e.g. amantadine), synthesis or translation of viral mRNA (e.g. interferon), replication of viral RNA or DNA (e.g. nucleoside analogues), maturation of new virus proteins (e.g. protease inhibitors), and budding and release of the virus.

Anti-fungal agents are useful for the treatment and prevention of infective fungi and parasiticides are agents that kill parasites directly. Such compounds are known in the art and are generally commercially available.

In addition to the use of the immunostimulatory nucleic acids and therapeutic formulations to prevent infection in humans, the methods of the preferred embodiments are particularly well suited for treatment of non-human vertebrates. Non-human vertebrates which exist in close quarters and which are allowed to intermingle as in the case of zoo, farm and research animals are also embraced as subjects for the methods of the invention. Zoo animals such as the felid species including for example lions, tigers, leopards, cheetahs, and cougars; elephants, giraffes, bears, deer, wolves, yaks, non-human primates, seals, dolphins and whales; and research animals such as mice, rats, hamsters and gerbils are all potential subjects for the methods of the invention.

Birds such as hens, chickens, turkeys, ducks, geese, quail, and pheasant are prime targets for many types of infections. Hatching birds are exposed to pathogenic microorganisms shortly after birth. Although these birds are initially protected against pathogens by maternal derived antibodies, this protection is only tenmporary, and the bird's own immature immune system must begin to protect the bird against the pathogens. It is often desirable to prevent infection in young birds when they are most susceptible. It is also desirable to prevent against infection in older birds, especially when the birds are housed in closed quarters, leading to the rapid spread of disease. Thus, it is desirable to administer the immunostimulatory nucleic acids and anti-microbial agents to birds to prevent infectious disease.

An example of a common infection in chickens is chicken infectious anemia virus (CIAV). CIAV was first isolated in Japan in 1979 during an investigation of a Marek's disease-vaccination break (Yuasa et al., 1979, Avian Dis. 23:366-385). Since that time, CIAV has been detected in commercial poultry in all major poultry producing countries (van Bulow et al., 1991, pp. 690-699) in Diseases of Poultry, 9th edition, Iowa State University Press).

CIAV infection results in a clinical disease, characterized by anemia, hemorrhage and immunosuppression, in young susceptible chickens. Atrophy of the thymus and of the bone marrow and consistent lesions of CIAV-infected chickens are also characteristic of CIAV infection. Lymphocyte depletion in the thymus, and occasionally in the bursa of Fabricius, results in immunosuppression and increased susceptibility to secondary viral, bacterial, or fugal infections which then complicate the course of the disease. The immunosuppression may cause aggravated disease after infection with one or more of Marek's disease virus (MDV), infectious bursal disease virus, reticuloendotheliosis virus, adenovirus, or reovirus. It has been reported that pathogenesis of MDV is enhanced by CIAV (DeBoer et aL, 1989, p. 28 In Proceedings of the 38th Western Poultry Diseases Conference, Tempe, Ariz.). Further, it has been reported that CIAV aggravates the signs of infectious bursal disease (Rosenberger et al., 1989, Avian Dis. 33:707-713). Chickens develop an age resistance to experimentally induced disease due to CAA. This is essentially complete by the age of 2 weeks, but older birds are still susceptible to infection (Yuasa, N. et al., 1979 supra; Yuasa, N. et al., Arian Diseases 24,202-209, 1980). However, if chickens are dually infected with CAA and an immunosuppressive agent (IBDV, MDV etc.) age resistance against the disease is delayed (Yuasa, N. et al., 1979 and 1980 supra; Bulow von V. et al., 3. Veterinary Medicine 33, 93-116, 1986). Characteristics of CIAV that may potentiate disease transmission include high resistance to environmental inactivation and some common disinfectants. The economic impact of CIAV infection on the poultry industry is clear from the fact that 10% to 30% of infected birds in disease outbreaks die.

Cattle and livestock are also susceptible to infection. Disease which affect these animals can produce severe economic losses, especially amongst cattle. The methods of the invention can be used to protect against infection in livestock, such as cows, horses, pigs, sheep, and goats.

Cows can be infected by bovine viruses. Bovine viral diarrhea virus (BVDV) is a small enveloped positive-stranded RNA virus and is classified, along with hog cholera virus (HOCV) and sheep border disease virus (BDV), in the pestivirus genus. Although, Pestiviruses were previously classified in the Togaviridae family, some studies have suggested their reclassification within the Flaviviridae family along with the flavivirus and hepatitis C virus (HCV) groups (Francki, et al., 1991).

BVDV, which is an important pathogen of cattle can be distinguished, based on cell culture analysis, into cytopathogenic (CP) and noncytopathogenic (NCP) biotypes. The NCP biotype is more widespread although both biotypes can be found in cattle. If a pregnant cow becomes infected with an NCP strain, the cow can give birth to a persistently infected and specifically immunotolerant calf that will spread virus during its lifetime. The persistently infected cattle can succumb to mucosal disease and both biotypes can then be isolated from the animal. Clinical manifestations can include abortion, teratogenesis, and respiratory problems, mucosal disease and mild diarrhea. In addition, severe thrombocytopenia, associated with herd epidemics, that may result in the death of the animal has been described and strains associated with this disease seem more virulent than the classical BVDVs.

Equine herpesviruses (EHV) comprise a group of antigenically distinct biological agents which cause a variety of infections in horses ranging from subclinical to fatal disease. These include Equine herpesvirus-1 (EHV-1), a ubiquitous pathogen in horses. EHV-1 is associated with epidemics of abortion, respiratory tract disease, and central nervous system disorders. Primary infection of upper respiratory tract of young horses results in a febrile illness which lasts for 8 to 10 days. Immunologically experienced mares may be reinfected via the respiratory tract without disease becoming apparent, so that abortion usually occurs without warning. The neurological syndrome is associated with respiratory disease or abortion and can affect animals of either sex at any age, leading to in-coordination, weakness and posterior paralysis (Telford, E. A. R. et al., Virology 189, 304-316, 1992). Other EHV's include EHV-2, or equine cytomegalovirus, EHV-3, equine coital exanthema virus, and EHV-4, previously classified as EHV-1 subtype 2.

Sheep and goats can be infected by a variety of dangerous microorganisms including visna-maedi.

Primates such as monkeys, apes and macaques can be infected by simian immunodeficiency virus. Inactivated cell-virus and cell-free whole simian immunodeficiency vaccines have been reported to afford protection in macaques (Stott et al. (1990) Lancet 36:1538-1541; Desrosiers et al. PNAS USA (1989) 86:6353-6357; Murphey-Corb et al. (1989) Science 246:1293-1297; and Carlson et al. (1990) AIDS Res. Human Retroviruses 6:1239-1246). A recombinant HIV gp120 vaccine has been reported to afford protection in chimpanzees (Berman et al. (1990) Nature 345:622-625).

Cats, both domestic and wild, are susceptible to infection with a variety of microorganisms. For instance, feline infectious peritonitis is a disease which occurs in both domestic and wild cats, such as lions, leopards, cheetahs, and jaguars. When it is desirable to prevent infection with this and other types of pathogenic organisms in cats, the methods of the invention can be used to prevent or treat infection in cats.

Domestic cats may become infected with several retroviruses, including but not limited to feline leukemia virus (FeLV), feline sarcoma virus (FeSV), endogenous type C oncornavirus (RD-14), and feline syncytia-forming virus (FeSFV). Of these, FeLV is the most significant pathogen, causing diverse symptoms, including lymphoreticular and myeloid neoplasms, anemias, immune mediated disorders, and an immunodeficiency syndrome which is similar to human acquired immune deficiency syndrome (AIDS). Recently, a particular replication-defective FeLV mutant, designated FeLV-AIDS, has been more particularly associated with immunosuppressive properties.

The discovery of feline T-lymphotropic lentivirus (also referred to as feline immunodeficiency) was first reported in Pedersen et al. (1987) Science 235:790-793. Characteristics of FIV have been reported in Yamamoto et al. (1988) Leukemia, December Supplement 2:204S-215S; Yamamoto et al. (1988) Am. J. Vet. Res. 49:1246-1258; and Ackley et al. (1990) J. Virol. 64:5652-5655. Cloning and sequence analysis of FIV have been reported in Olmsted et al. (1989) Proc. Natl. Acad. Sci. USA 86:2448-2452 and 86:4355-4360.

Feline infectious peritonitis (FIP) is a sporadic disease occurring unpredictably in domestic and wild Felidae. While FIP is primarily a disease of domestic cats, it has been diagnosed in lions, mountain lions, leopards, cheetahs, and the jaguar. Smaller wild cats that have been afflicted with FIP include the lynx and caracal, sand cat, and pallas cat. In domestic cats, the disease occurs predominantly in young animals, although cats of all ages are susceptible. A peak incidence occurs between 6 and 12 months of age. A decline in incidence is noted from 5 to 13 years of age, followed by an increased incidence in cats 14 to 15 years old.

Viral, bacterial, and parasitic diseases in fin-fish, shelfish or other aquatic life forms pose a serious problem for the aquaculture industry. Owing to the high density of animals in the hatchery tanks or enclosed marine farming areas, infectious diseases may eradicate a large proportion of the stock in, for example, a fin-fish, shellfish, or other aquatic life forms facility. The fish immune system has many features similar to the mammalian immune system, such as the presence of B cells, T cells, lymphokines, complement, and immunoglobulins. Fish have lymphocyte subclasses with roles that appear similar in many respects to those of the B and T cells of mammals.

Aquaculture species include but are not limited to fin-fish, shellfish, and other aquatic animals. Fin-fish include all vertebrate fish, which may be bony or cartilaginous fish, such as, for example, salmonids, carp, catfish, yellowtail, seabream, and seabass. Salmonids are a family of fin-fish which include trout (including rainbow trout), salmon, and Arctic char. Examples of shellfish include, but are not limited to, clams, lobster, shrimp, crab, and oysters. Other cultured aquatic animals include, but are not limited to eels, squid, and octopi.

In some cases it is desirable to administer an antigen with the immunostimulatory nucleic acid and the therapeutic formulations and in other cases no antigen is delivered. The antigen, if used, is preferably a microbial antigen. Microbial antigens include, but are not limited to, cells, cell extracts, proteins, polypeptides, peptides, polysaccharides, polysaccharide conjugates, peptide and non-peptide mimics of polysaccharides and other molecules, small molecules, lipids, glycolipids, and carbohydrates. Many microbial antigens, however, are protein or polypeptide in nature, as proteins and polypeptides are generally more antigenic than carbohydrates or fat. Methods for administer an antigen to a subject are well-known in the art. In general, an antigen is administered directly to the subject by any means, such as, e.g., intravenous, intramuscular, oral, transdermal, mucosal, intranasal, intratracheal, or subcutaneous administration. The antigen can be administered systemically or locally. In some preferred embodiments, the antigen is not conjugated to the immunostimulatory nucleic acid. Administration methods are described in more detail below.

The term “substantially purified” as used herein refers to a molecular species which is substantially free of other proteins, lipids, carbohydrates or other materials with which it is naturally associated. One skilled in the art can purify polypeptides, e.g. antigens, using standard techniques for protein purification The substantially pure polypeptide will often yield a single major band on a non-reducing polyacrylamide gel. In the case of partially glycosylated polypeptides or those that have several start codons, there may be several bands on a non-reducing polyacrylamide gel, but these will form a distinctive pattern for that polypeptide. The purity of the polypeptide can also be determined by amino-termial amino acid sequence analysis.

The microbial antigen, if administered and if it is a polypeptide, may be in the form of a polypeptide when administered to the subject or it may be encoded by a nucleic acid vector. If the nucleic acid vector is administered to the subject the protein is expressed in vivo. Minor modifications of the primary amino acid sequences of polypeptide microbial antigens may also result in a polypeptide which has substantially equivalent antigenic activity, as compared to the unmodified counterpart polypeptide. Such modifications may be deliberate, as by site-directed mutagenesis, or may be spontaneous. Thus, nucleic acids having such modifications are also encompassed. When an antigen that is encoded by a nucleic acid vector is administered, the immunostimulatory nucleic acid is not the same plasmid or expression vector containing the antigen.

The nucleic acid encoding the antigen is operatively linked to a gene expression sequence which directs the expression of the protein within a eukaryotic cell. The “gene expression sequence” is any regulatory nucleotide sequence, such as a promoter sequence or promoter-enhancer combination, which facilitates the efficient transcription and translation of the protein which it is operatively linked. The gene expression sequence may, for example, be a mammalian or viral promoter, such as a constitutive or inducible promoter. Constitutive mammalian promoters include, but are not limited to, the promoters for the following genes: hypoxanthine phosphonbosyl transferase (HPTR), adenosine deaminase, pyruvate kinase, b-actin promoter and other constitutive promoters. Exemplary viral promoters which function constitutively in eukaryotic cells include, for example, promoters from the cytomegalovirus (CMV), simian virus (e.g., SV40), papilloma virus, adenovirus, human immunodeficiency virus (HIV), Rous sarcoma virus, cytomegalovirus, the long terminal repeats (LTR) of Moloney leukemia virus and other retroviruses, and the thymidine kinase promoter of herpes simplex virus. Other constitutive promoters are known to those of ordinary skill in the art. The promoters useful as gene expression sequences of the invention also include inducible promoters. Inducible promoters are expressed in the presence of an inducing agent. For example, the metallothionein promoter is induced to promote transcription and translation in the presence of certain metal ions. Other inducible promoters are known to those of ordinary skill in the art.

The combination of immunostimulatory nucleic and therapeutic formulations is also useful for treating and preventing cancer. Present cancer treatments are too often ineffective as well as being associated with a high degree of patient morbidity, most probably due to a lack of toxic specificity for tumor cells. The compositions of the invention provide a more effective treatment of cancer by promoting an enhanced immune response. The immune response may be antigen specific or an innate immune response (non-antigen specific). In some instances, the combination of the immunostimulatory nucleic acid and therapeutic formulations is synergistic, resulting in greater than additive effects than would otherwise be expected using the agents separately.

Thus, in one aspect, the invention provides a method for treating or preventing cancer which involves the administration of some forms of immunostimulatory nucleic acid and some forms of the therapeutic formulations in an effective amount to prevent or treat the cancer to a subject having cancer or a subject at risk of developing cancer.

A cancer cell is a cell that divides and reproduces abnormally due to a loss of normal growth control. Cancer cells almost always arise from at least one genetic mutation. In some instances, it is possible to distinguish cancer cells from their normal counterparts based on profiles of expressed genes and proteins, as well as to the level of their expression. Genes commonly affected in cancer cells include oncogenes, such as ras, neu/HER2/erbB, myb, myc and abl, as well as tumor suppressor genes such as p53, Rb, DCC, RET and WT. Cancer-related mutations in some of these genes leads to a decrease in their expression or a complete deletion. In others, mutations cause an increase in expression or the expression of an activated variant of the normal counterpart. Genetic mutations in cancer cells can be targets of therapeutic formulations in some instances. For example, some medicaments target proteins which are thought to be necessary for cancer cell survival and division, such as cell cycle proteins (e.g., cyclin dependent kinases), telomerase and telomerase associated proteins, and tumor suppressor proteins, many of which are upregulated, or unregulated, in cancer cells.

The term “tumor” is usually equated with neoplasm, which literally means “new growth” and is used interchangeably with “cancer.” A “neoplastic disorder” is any disorder associated with cell proliferation, specifically with a neoplasm. A “neoplasm” is an abnormal mass of tissue that persists and proliferates after withdrawal of the carcinogenic factor that initiated its appearance. There are two types of neoplasms, benign and malignant. Nearly all benign tumors are encapsulated and are noninvasive; in contrast malignant tumors are almost never encapsulated but invade-adjacent tissue by infiltrative destructive growth. This infiltrative growth can be followed by tumor cells implanting at sites discontinuous with the original tumor. The method of the invention can be used to treat neoplastic disorders in humans, including but not limited to: sarcoma, carcinoma, fibroma, leukemia, lymphoma, melanoma, myeloma, neuroblastoma, rhabdomyosarcoma, retinoblastoma, and glioma as well as each of the other tumors described herein.

“Cancer” as used herein refers to an uncontrolled growth of cells which interferes with the normal functioning of the bodily organs and systems. Cancers which migrate from their original location and seed vital organs can eventually lead to the death of the subject through the functional deterioration of the affected organs. Hemopoietic cancers, such as leukemia, are able to outcompete the normal hemopoietic compartments in a subject, thereby leading to hemopoietic failure (in the form of anemia, thrombocytopenia and neutropenia) ultimately causing death.

A metastasis is a region of cancer cells, distinct from the primary tumor location resulting from the dissemination of cancer cells from the primary tumor to other parts of the body. At the time of diagnosis of the primary tumor mass, the subject may be monitored for the presence of metastases. Metastases are most often detected through the sole or combined use of magnetic resonance imaging (MRI) scans, computed tomography (CT) scans, blood and platelet counts, liver function studies, chest X-rays and bone scans in addition to the monitoring of specific symptoms.

Cancers include, but are not limited to, basal cell carcinoma, biliary tract cancer; bladder cancer, bone cancer; brain and CNS cancer; breast cancer; cervical cancer; choriocarcinoma; colon and receen cancer; connective tissue cancer, cancer of the digestive system; endometrial cancer, esophageal cancer, eye cancer, cancer of the head and neck; gastric cancer, intra-epithelial neoplasm; kidney cancer, larynx cancer, leukemia; liver cancer; lung cancer (e.g. small cell and non-small cell); lymphoma including Hodgkin's and Non-Hodgkin's lymphoma; melanoma; myeloma; neuroblastoma; oral cavity cancer (e.g., lip, tongue, mouth, and pharnx); ovarian cancer, pancreatic cancer; prostate cancer, retinoblastoma; rhabdomyosarcoma; rectal cancer, renal cancer, cancer of the respirtory system; sarcoma; skin cancer, stomach cancer, testicular cancer, thyroid cancer; uterine cancer; cancer of the urinary system, as well as other carcinomas and sarcomas.

The immunostimulatory nucleic acids and therapeutic formulations are useful for treating or preventing cancer in a subject. The invention can be used to treat cancer and tumors in human and non human subjects. Cancer is one of the leading causes of death in companion animals (i.e., cats and dogs). Cancer usually strikes older animals which, in the case of house pets, have become integrated into the family. Forty-five % of dogs older than 10 years of age, are likely to succumb to the disease. The most common treatment options include surgery, chemotherapy and radiation therapy. Others treatment modalities which have been used with some success are laser therapy, cryotherapy, hyperthermia and immunotherapy. The choice of treatment depends on type of cancer and degree of dissemination. Unless the. malignant growth is confined to a discrete area in the body, it is difficult to remove only malignant tissue without also affecting normal cells.

Malignant disorders commonly diagnosed in dogs and cats include but are not limited to lymphosarcoma, osteosarcoma, mammary tumors, mastocytoma, brain tumor, melanoma, adenosquamous carcinoma, carcinoid lung tumor, bronchial glaid tumor, bronchiolar adenocarcinoma, fibroma, myxochondroma, pulmonary sarcoma, neurosarcoma, osteoma, papilloma, retinoblastoma, Ewing's sarcoma, Wilm's tumor, Burkitt's lymphoma, microglioma, neuroblastoma, osteoclastoma, oral neoplasia, fibrosarcoma, osteosarcoma and rhabdomyosarcoma. Other neoplasias in dogs include genital squamous cell carcinoma, transmissable veneral tumor, testicular tumor, seminoma, Sertoli cell tumor, hemangiopericytoma, histiocytoma, chloroma (granulocytic sarcoma), corneal papilloma, corneal squamous cell carcinoma, hemangiosarcoma, pleural mesothelioma, basal cell tumor, thymoma, stomach tumor, adrenal gland carcinoma, oral papillomatosis, hemangioendothelioma and cystadenoma Additional malignancies diagnosed in cats include follicular lymphoma, intestinal lymphosarcoma, fibrosarcoma and pulmonary squamous cell carcinoma The ferret, an ever-more popular house pet, is knlown to develop insulinoma, lymphoma, sarcoma, neuroma, pancreatic islet cell tumor, gastric MALT lymphoma and gastric adenocarcinoma.

Neoplasias affecting agricultural livestock include leukemia, hemangiopericytoma and bovine ocular neoplasia (in cattle); preputial fibrosarcomna, ulcerative squamous cell carcinoma, preputial carcinoma, connective tissue neoplasia and mastocytoma (in horses); hepatocellular carcinoma (in swine); lymphoma and pulmonary adenomatosis (in sheep); pulmonary sarcoma, lymphoma, Rous sarcoma, reticulo-endotheliosis, fibrosarcoma, nephroblastoma, B-cell lymphoma and lymphoid leukosis (in avian species); retinoblastoma, hepatic neoplasia, lymphosarcoma (lymphoblastic lymphoma), plamracytoid leukemia and swimbladder sarcoma (in fish), caseous lumphadenitis (CLA): chronic, infectious, contagious disease of sheep and goats caused by the bacterium Corynebacterium pseudotuberculosis, and contagious lung tumor of sheep caused by jaagsiekte.

In one aspect, a method for treating cancer is provided which involves administering the compositions of the invention to a subject having cancer. A “subject having cancer” is a subject that has been diagnosed with a cancer. In some embodiments, the subject has a cancer type characterized by a solid mass tumpor. The solid tumor mass, if present, may be a primary tumor mass. A primary tumor mass refers to a growth of cancer cells in a tissue resulting from the transformation of a normal cell of that tissue. In most cases, the primary tumor mass is identified by the presence of a cyst, which can be found through visual or palpation methods, or by irregularity in shape, texture or weight of the tissue.

However, some primary-tumors are not palpable and can be detected only through medical imaging techniques such as X-rays (e.g., mammography), or by needle aspirations. The use of these latter techniques is more common in early detection. Molecular and phenotypic analysis of cancer cells within a tissue will usually confirm if the cancer is endogenous to the tissue or if the lesion is due to metastasis from another site.

With respect to the prophylactic treatment methods, the invention is aimed at administering the compositions of the invention to a subject at risk of developing cancer. A subject at risk of developing a cancer is one who has a high probability of developing cancer. These subjects include, for instance, subjects having a genetic abnormality, the presence of which has been demonstrated to have a correlative relation to a higher likelihood of developing a cancer. Subjects exposed to cancer causing agents such as tobacco, asbestos, or other chemical toxins are also subjects at risk of developing cancers used herein. When a subject at risk of developing a cancer is treated with an immunostimulatory nucleic acid and therapeutic formulations, on a regular basis, such as monthly, the subject will be able to mount a continuous immune response against the cancer. An antigen may also be used to provoke a cancer specific immune response. If a tumor begins to form in the subject, the subject will develop a specific immune response against one or more of the cancer antigens. This aspect of the invention is particularly advantageous when the antigen to which the subject will be exposed is known. For instance, subjects employed in certain trades which are exposed to cancer-causing agents on an ongoing basis would be ideal subjects for treatment according to the invention, particularly because cancer-causing agents usually preferentially target a specific organ or tissue. For example, many air borne, or inhaled, carcinogens such as tobacco smoke and asbestos have been associated with lung cancer. The methods in which a subject is passively exposed to an carcinogen can be particularly dependent on timing of the administration of the immunostimulatory nucleic acid and the therapeutic formulation, preferably in the form of a cancer vaccine (e.g., a cancer antigen). For instance, in a subject at risk of developing a cancer, the subject may be administered the immunostimulatory nucleic acid and the cancer vaccine containing a cancer antigen on a regular basis when that risk is greatest, i.e., after exposure to a cancer causing agent.

The immunostimulatory nucleic acid and therapeutic formulation may also be administered in combination with a cancer medicament. As used herein, a “cancer medicament” refers to a agent which is administered to a subject for the purpose of treating a cancer. As used herein, “seating cancer” includes preventing the development of a cancer, reducing the symptoms of cancer, and/or inhibiting the growth of an established cancer. In other aspects, the cancer medicament is administered to a subject at risk of developing a cancer for the purpose of reducing the risk of developing the cancer. Cancer medicaments embrace such categories as chemotherapeutic agents, immunotherapeutic agents, cancer vaccines, hormone-therapy, and biological response modifiers. Cancer medicaments also include agents which are administered to a subject in order to reduce the symptoms of a cancer, rather than to reduce the tumor or cancer burden (i.e., the number of cancer or tumor cells) in a subject. One example of this latter type of cancer medicament is a blood transfusion which is administered to a subject having cancer in order to maintain red blood cell and/or platelet levels within a normal range. As an example, in the absence of such transfusion, cancer patients with below normal levels of platelets are at risk of uncontrolled bleeding.

As used herein a cancer antigen is broadly defined as an antigen expressed by a cancer cell. Preferably, the antigen is expressed at the cell surface of the cancer cell. Even more preferably, the antigen is one which is not expressed by normal cells, or at least not expressed to the same level as in cancer cells. For example, some cancer antigens are normally silent (i.e., not expressed) in normal cells, some are expressed only at certain stages of differentiation and others are temporally expressed such as embryonic and fetal antigens. Other cancer antigens are encoded by mutant cellular genes, such as oncogenes (e.g., activated ras oncogene), suppressor genes (e.g., mutant p53), fusion proteins resulting from internal deletions or chromosomal translocations. Still other cancer antigens can be encoded by viral genes such as those carried on RNA and DNA tumor viruses. The differential expression of cancer antigens in normal and cancer cells can be exploited in order to target cancer cells. As used herein, the terms “cancer antigen” and “tumor antigen” are used interchangeably.

In other aspects of the invention, the use of immunostimulatory nucleic acids, either alone or in combination with the therapeutic formulations, allows for the administration of lower doses of antigen than could ordinarily be administered to produce an effective antigen specific immune response. Thus, the immunostimulatory nucleic acids allow for the administration of lower, sub-therapeutic doses of the antigen, but with higher efficacy than would otherwise be achieved using such low doses. As one example, by administering an immunostimulatory nucleic acid with a dose of antigen that if otherwise used in combination with a conventional adjuvant such as alum would be ineffective, it is possible to achieve an effective immune response against the antigen even though one of skill in the art would not have expected that dose of antigen to provide a therapeutic benefit (i.e., a sub-therapeutic dose).

An “immunostimulatory nucleic acid” as used herein is any nucleic acid containing an immunostimulatory motif or backbone that induces an immune response. The immune response may be characterized as, but is not limited to, a Th1-type immune response or a Th2-type immune response. Such immune responses are defined by cytokine and antibody production profiles which are elicited by the activated immune cells.

Helper (CD4⁺) T cells orchesrate the immune response of mammals through production of soluble factors that act on other immune system cells, including other T cells. Helper CD4⁺, and in some instances also CD8⁺, T cells are characterized as Th1 and Th2 cells in both murine and human systems, depending on their cytokine production profiles (Romagnani 1991, Immunol Today 12: 256257, Mosmann, 1989, Annu Rev Immunol, 7: 145-173). Th1 cells produce interleukin 2 (IL-2), II-12, tumor necrosis factor (TNFα) and interferon gamma (FN-γ) and they are responsible primarily for cell-mediated immunity such as delayed type hypersensitivity. The cytokines that are induced by administration of immunostimulatory nucleic acids are predominantly of the Th1 class. The types of antibodies associated with a Th1 response are generally more protective because they have high neutralization and opsonization capabilities. Th2 cells produce IL-4, IL-5, IL-6, IL-9, IL-10 and IL-13 and are primarily involved in providing optimal help for humoral immune responses such as IgE and IgG4 antibody isotype switching (Mosmann 1989, Annu Rev Immunol, 7: 145-173). Th2 responses involve predominantly antibodies that have less protective effects against infection.

The terms “nucleic acid” and “oligonucleotide” are used interchangeably to mean multiple nucleotides (i.e. molecules comprising a sugar (e.g. ribose or deoxyribose) linked to a phosphate group and to an exchangeable organic base, which is either a substituted is pyrimidine (e.g. cytosine (C), thymine (T) or uracil (U)) or a substituted purine (e.g. adenine (A) or guanine (G)). As used herein, the terms refer to oligoribonucleotides as well as oligodeoxyribonucleotides. The terms shall also include polynucleosides (i.e. a polynucleotide minus the phosphate) and any other organic base containing polymer. Nucleic acids include vectors, e.g., plasmids, as well as oligonucleotides. Nucleic acid molecules can be obtained from existing nucleic acid sources (e.g., genomic or cDNA, referred to as isolated nucleic acids), but are preferably synthetic (e.g. produced by oligonucleotide synthesis).

Immunostimulatory nucleic acids may possess immunostimulatory motifs such as CpG motif; and poly-G motifs. In some embodiments of the invention, any nucleic acid, regardless of whether it possesses an identifiable motif, can be used in the combination therapy to elicit an immune response. Immunostimulatory backbones include, but are not limited to, phosphate modified backbones, such as phosphorothioate backbones. Immunostimulatory nucleic acids have been described extensively in the prior art and a brief summary of these nucleic acids is presented below. Most aspects of the invention, particularly those directed at treating subjects having or at risk of developing cancer, do not embrace the use of T-rich or methylated CpG nucleic acids (i.e., nucleic acids that possess either a T-rich or a methylated CpG motif).

In some embodiments, a CpG immunostimulatory nucleic acid is used in the methods of the invention. A CpG immunostimulatory nucleic acid is a nucleic acid which contains a CG dinucleotide, the C residue of which is unmethylated. CpG immunostimulatory nucleic acids are known to stimulate Th1-type immune responses. CpG sequences, while relatively rare in human DNA are commonly found in the DNA of infectious organisms such as bacteria. The human immune system has apparently evolved to recognize CpG sequences as an early warning sign of infection and to initiate an immediate and powerful immune response against invading pathogens without causing adverse reactions frequently seen with other immune stimulatory agents. Thus CpG containing nucleic acids, relying on this innate immune defense mechanism can utilize a unique and natural pathway for immune therapy. The effects of CpG nucleic acids on immune modulation have been described extensively in U.S. Pat. No. 6,194,388, and published patent applications, such as PCT US95/01570, PCT/US97/19791, PCT/US98/03678, PCT/US98/10408, PCI/US98/04703, PCT/US99/07335, and PCT/US99/09863. The entire contents of each of these issued patents and patent applications are hereby incorporated by reference.

A CpG nucleic acid is a nucleic acid which includes at least one unmethylated CpG dinucleotide. A nucleic acid containing at least one unmethylated CpG dinucleotide is a nucleic acid molecule which contains an methylated cytosine in a cytosine-guanine dinucleotide sequence (i.e. “CpG DNA” or DNA containing a 5′ cytosine followed by 3′ guanosine and linked by a phosphate bond) and activates the immune system. The CpG nucleic acids can be double-stranded or single-stranded. Generally, double-stranded molecules are more stable in vivo, while single-stranded molecules have increased immune activity. Thus in some aspects of the invention it is preferred that the nucleic acid be single stranded and in other aspects it is preferred that the nucleic acid be double stranded The terms CpG nucleic acid or CpG oligonucleotide as used herein refer to an immunostimulatory CpG nucleic acid unless otherwise indicated. The entire immunostimulatory nucleic acid can be unmethylated or portions may be unmethylated but at least the C of the 5′ CG 3′ must be unmethylated.

In one preferred embodiment the invention provides an immunostimulatory nucleic acid which is a C-pG nucleic acid represented by at least the formula:
5′X₁X₂CGX₃X₄3′
wherein X₁, X₂, X₃, and X₄ are nucleotides. In one embodiment X₂ is adenine, guanine, cytosine, or thymine. In another embodiment X₃ is cytosine, guanine, adenine, or thymine. In other embodiments X₂ is adenine, guanine, or thymine and X₃ is cytosine, adenine, or thymine.

In another embodiment the immunostimulatory nucleic acid is an isolated CpG nucleic acid represented by at least the formula:
5′N₁X₁X₂CGX₃X₄N₂3′
wherein X₁, X₂, X₃, and X₄ are nucleotides and N is any nucleotide and N₁ and N₂ are nucleic acid sequences composed of from about 0-25 N's each. In one embodiment X₁X₂ are nucleotides selected from the group consisting of: GpT, GpG, GpA, ApA, ApT, ApG, CpT, CpA, CpG, TpA, TpT, and TpG; and X₃X₄ are nucleotides selected from the group consisting of: TpT, ApT, TpG, ApG, CpG, TpC, ApC, CpC TpA, ApA, and CpA. Preferably X₁X₂ are GpA or GpT and X₃X₄ are TpT. In other embodiments X₁ or X₂ or both are purines and X₃ or X₄ or both are pyrimidines or X₁X₂ are GpA and X₃ or X₄ or both are pyrimidines. In another preferred embodiment X₁X₂ are nucleotides selected from the group consisting of: TpA, ApA, ApC, ApG, and GpG. In yet another embodiment X₃X₄ are nucleotides selected from the group consisting of: TpT, TpA, TpG, ApA, ApG, ApC, and CpA. X₁X₂ in another embodiment are nucleotides selected from the group consisting of: TpT, TpG, ApT, GpC, CpC, CpT, TpC, GpT and CpG.

In another preferred embodiment the immunostimulatory nucleic acid has the sequence 5′TCN₁TX₁X₂CGX₃X₄3′. The immunostimulatory nucleic acids of the invention in some embodiments include X₁X₂ selected from the group consisting of GpT, GpG, GpA and ApA and X₃X₄ is selected from the group consisting of TpT, CpT and TpC.

For facilitating uptake into cells, the immunostimulatory nucleic acids are preferably in the range of 6 to 100 bases in length However, nucleic acids of any size greater than 6 nucleotides (even many kb long) are capable of inducing an immune response according to the invention if sufficient immunostimulatory motifs are present. Preferably the immunostimulatory nucleic acid is in the range of between 8 and 100 and in some embodiments between 8 and 50 or 8 and 30 nucleotides in size.

“Palindromic sequence” shall mean an inverted repeat (i.e., a sequence such as ABCDEE′D′C′B′A′ in which A and A′ are bases capable of forming the usual Watson-Crick base pairs). In vivo, such sequences may form double-stranded structures. In one embodiment the CpG nucleic acid contains a palindromic sequence. A palindromic sequence used in this context refers to a palindrome in which the CpG is part of the palindrome, and preferably is the center of the palindrome. In another embodiment the CpG nucleic acid is free of a palindrome. An immunostimulatory nucleic: acid that is free of a palindrome is one in which the CpG dinucleotide is not part of a palindrome. Such an Oligonucleotide may include a palindrome in which the CpG is not the center of the palindrome.

In some embodiments of the invention, a non-CpG immunostimulatory nucleic acid is used. A non-CpG immunostimulatory nucleic acid is a nucleic acid which does not have a CpG motif in its sequence, regardless of whether the C is the dinucleotide is methylated or unmethylated. Non-CpG immunostimulatory nucleic acids may induce Th1 or Th2 immune responses, depending upon their sequence, their mode of delivery and the dose at which they are administered.

An important subset of non-CpG immunostimulatory nucleic acids are poly-G immunostimulatory nucleic acids. A variety of references, including Pisetsky and Reich, 1993 Mol. Biol. Reports, 18:217-221; Krieger and Herz, 1994, Ann. Rev. Biochem., 63:601-637; Macaya et al., 1993, PNAS, 90:3745-3749; Wyatt et al., 1994, PNAS, 91:1356-1360; Rando and Hogan, 1998, In Applied Antisense Oligonucleotide Technology, ed. Krieg and Stein, p. 335-352; and Kimura et al., 1994, J. Biochem. 116, 991-994 also describe the immunostimulatory properties of poly-G nucleic acids. In accordance with one aspect of the invention, poly-G-containing nucleotides are useful, inter alia, for treating and preventing bacterial, viral and fungal infections, and can thereby be used to minimize the impact of these infections on the treatment of cancer patients.

Poly-G nucleic acids preferably are nucleic acids having the following formulas:
5′ X₁X₂GGGX₃X₄3′
wherein X₁, X₂, X₃, and X₄ are nucleotides. In preferred embodiments at least one of X₃ and X₄ are a G. In other embodiments both of X₃ and X₄ are a G. In yet other embodiments the preferred formula is 5′ GGGNGGG3′, or 5′GGGNGGGNGGG3′ wherein N represents between 0 and 20 nucleotides. In other embodiments the Poly-G nucleic acid is free of unmethylated CG dinucleotides, such as, for example, the nucleic acids listed above as SEQ ID NO: 95 through to SEQ ID NO: 133. In other embodiments the Poly-G nucleic acid includes at least one unmethylated CG dinucleotide, such as, for example, the nucleic acids listed below as SEQ ID NO: 46, SEQ ID NO: 47, SEQ ID NO: 58, and SEQ ID NO: 61.

T-rich motifs and nucleic acids possessing such motifs are described in Published PCT patent application WO 01/22972 and related U.S. patent application Ser. No. 09/669,187 filed Sep. 25, 2000, the entire contents of which are incorporated herein by reference.

Exemplary immunostimulatory nucleic acid sequences include but are not limited to those immunostimulatory sequences shown in Table 1.

TABLE 1

| | |
|---|---|
| GCTAGACGTTAGCGT; | (SEQ ID NO: 1) |
| GCTAGATGTTAGCGT; | (SEQ ID NO: 2) |
| GCTAGACGTTAGCGT; | (SEQ ID NO: 3) |
| GCTAGACGTTAGCGT; | (SEQ ID NO: 4) |
| GCATGACGTTGAGCT; | (SEQ ID NO: 5) |
| ATGGAAGGTCCAGCGTTCTC; | (SEQ ID NO: 6) |
| ATCGACTCTCGAGCGTTCTC; | (SEQ ID NO: 7) |
| ATCGACTCTCGAGCGTTCTC; | (SEQ ID NO: 8) |
| ATCGACTCTCGAGCGTTCTC; | (SEQ ID NO: 9) |
| ATGGAAGGTCCAACGTTCTC; | (SEQ ID NO: 10) |
| GAGAACGCTGGACCTTCCAT; | (SEQ ID NO: 11) |
| GAGAACGCTCGACCTTCCAT; | (SEQ ID NO: 12) |
| GAGAACGCTCGACCTTCGAT; | (SEQ ID NO: 13) |
| GAGAACGCTGGACCTTCCAT; | (SEQ ID NO: 14) |
| GAGAACGATGGACCTTCCAT; | (SEQ ID NO: 15) |
| GAGAACGCTCCAGCACTGAT; | (SEQ ID NO: 16) |
| TCCATGTCGGTCCTGATGCT; | (SEQ ID NO: 17) |
| TCCATGTCGGTCCTGATGCT; | (SEQ ID NO: 18) |
| TCCATGACGTTCCTGATGCT; | (SEQ ID NO: 19) |
| TCCATGTCGGTCCTGCTGAT; | (SEQ ID NO: 20) |
| TCAACGTT; | (SEQ ID NO: 21) |
| TCAGCGCT; | (SEQ ID NO: 22) |
| TCATCGAT; | (SEQ ID NO: 23) |
| TCTTCGAA; | (SEQ ID NO: 24) |
| CAACGTT; | (SEQ ID NO: 25) |
| CCAACGTT; | (SEQ ID NO: 26) |
| AACGTTCT; | (SEQ ID NO: 27) |
| TCAACGTC; | (SEQ ID NO: 28) |
| ATGGACTCTCCAGCGTTCTC; | (SEQ ID NO: 29) |
| ATGGAAGGTCCAACGTTCTC; | (SEQ ID NO: 30) |
| ATCGACTCTCGAGCGTTCTC; | (SEQ ID NO: 31) |
| ATGGAGGCTCCATCGTTCTC; | (SEQ ID NO: 32) |
| ATCGACTCTCGAGCGTTCTC; | (SEQ ID NO: 33) |
| ATCGACTCTCGAGCGTTCTC; | (SEQ ID NO: 34) |
| TCCATGTCGGTCCTGATGCT; | (SEQ ID NO: 35) |
| TCCATGCCGGTCCTGATGCT; | (SEQ ID NO: 36) |
| TCCATGGCGGTCCTGATGCT; | (SEQ ID NO: 37) |
| TCCATGACGGTCCTGATGCT; | (SEQ ID NO: 38) |
| TCCATGTCGATCCTGATGCT; | (SEQ ID NO: 39) |
| TCCATGTCGCTCCTGATGCT; | (SEQ ID NO: 40) |
| TCCATGTCGTCCCTGATGCT; | (SEQ ID NO: 41) |
| TCCATGACGTGCCTGATGCT; | (SEQ ID NO: 42) |
| TCCATAACGTTCCTGATGCT; | (SEQ ID NO: 43) |
| TCCATGACGTCCCTGATGCT; | (SEQ ID NO: 44) |
| TCCATCACGTGCCTGATGCT; | (SEQ ID NO: 45) |
| GGGGTCAACGTTGACGGGG; | (SEQ ID NO: 46) |
| GGGGTCAGTCGTGACGGGG; | (SEQ ID NO: 47) |
| GCTAGACGTTAGTGT; | (SEQ ID NO: 48) |
| TCCATGTCGTTCCTGATGCT; | (SEQ ID NO: 49) |
| ACCATGGACGATCTGTTTCCCCTC; | (SEQ ID NO: 50) |
| TCTCCCAGCGTGCGCCAT; | (SEQ ID NO: 51) |
| ACCATGGACGAACTGTTTCCCCTC; | (SEQ ID NO: 52) |
| ACCATGGACGAGCTGTTTCCCCTC; | (SEQ ID NO: 53) |
| ACCATGGACGACCTGTTTCCCCTC; | (SEQ ID NO: 54) |
| ACCATGGACGTACTGTTTCCCCTC; | (SEQ ID NO: 55) |
| ACCATGGACGGTCTGTTTCCCCTC; | (SEQ ID NO: 56) |
| ACCATGGACGTTCTGTTTCCCCTC; | (SEQ ID NO: 57) |
| CACGTTGAGGGGCAT; | (SEQ ID NO: 58) |
| TCAGCGTGCGCC; | (SEQ ID NO: 59) |
| ATGACGTTCCTGACGTT; | (SEQ ID NO: 60) |
| TCTCCCAGCGGGCGCAT; | (SEQ ID NO: 61) |
| TCCATGTCGTTCCTGTCGTT; | (SEQ ID NO: 62) |
| TCCATAGCGTTCCTAGCGTT; | (SEQ ID NO: 63) |
| TCGTCGCTGTCTCCCCTTCTT; | (SEQ ID NO: 64) |
| TCCTGACGTTCCTGACGTT; | (SEQ ID NO: 65) |
| TCCTGTCGTTCCTGTCGTT; | (SEQ ID NO: 66) |
| TCCATGTCGTTTTTGTCGTT; | (SEQ ID NO: 67) |
| TCCTGTCGTTCCTTGTCGTT; | (SEQ ID NO: 68) |
| TCCTTGTCGTTCCTGTCGTT; | (SEQ ID NO: 69) |
| TCCTGTCGTTTTTTGTCGTT; | (SEQ ID NO: 70) |
| TCGTCGCTGTCTGCCCTTCTT; | (SEQ ID NO: 71) |
| TCGTCGCTGTTGTCGTTTCTT; | (SEQ ID NO: 72) |
| TCCATGCGTGCGTGCGTTTT; | (SEQ ID NO: 73) |
| TCCATGCGTTGCGTTGCGTT; | (SEQ ID NO: 74) |
| TCCACGACGTTTTCGACGTT; | (SEQ ID NO: 75) |
| TCGTCGTTGTCGTTGTCGTT; | (SEQ ID NO: 76) |
| TCGTCGTTTTGTCGTTTTGTCGTT; | (SEQ ID NO: 77) |
| TCGTCGTTGTCGTTTTGTCGTT; | (SEQ ID NO: 78) |
| GCGTGCGTTGTCGTTGTCGTT; | (SEQ ID NO: 79) |
| TGTCGTTTGTCGTTTGTCGTT; | (SEQ ID NO: 80) |
| TGTCGTTGTCGTTGTCGTTGTCGTT; | (SEQ ID NO: 81) |
| TGTCGTTGTCGTTGTCGTT; | (SEQ ID NO: 82) |
| TCGTCGTCGTCGTT; | (SEQ ID NO: 83) |
| TGTCGTTGTCGTT; | (SEQ ID NO: 84) |
| TCCATAGCGTTCCTAGCGTT; | (SEQ ID NO: 85) |
| TCCATGACGTTCCTGACGTT; | (SEQ ID NO: 86) |
| GTCGYT; | (SEQ ID NO: 87) |
| TGTCGYT; | (SEQ ID NO: 88) |
| AGCTATGACGTTCCAAGG; | (SEQ ID NO: 89) |
| TCCATGACGTTCCTGACGTT; | (SEQ ID NO: 90) |
| ATCGACTCTCGAACGTTCTC; | (SEQ ID NO: 91) |
| TCCATGTCGGTCCTGACGCA; | (SEQ ID NO: 92) |
| TCTTCGAT; | (SEQ ID NO: 93) |
| ATGAGGAGGTCCAACGTTCTC; | (SEQ ID NO: 94) |
| GCTAGAGGGGAGGGT; | (SEQ ID NO: 95) |
| GCTAGATGTTAGGGG; | (SEQ ID NO: 96) |
| GCTAGAGGGGAGGGT; | (SEQ ID NO: 97) |
| GCTAGAGGGGAGGGT; | (SEQ ID NO: 98) |
| GCATGAGGGGGAGCT; | (SEQ ID NO: 99) |
| ATGGAAGGTCCAGGGGGCTC; | (SEQ ID NO: 100) |
| ATGGACTCTGGAGGGGGCTC; | (SEQ ID NO: 101) |
| ATGGACTCTGGAGGGGGCTC; | (SEQ ID NO: 102) |
| ATGGACTCTGGAGGGGGCTC; | (SEQ ID NO: 103) |
| ATGGAAGGTCCAAGGGGCTC; | (SEQ ID NO: 104) |
| GAGAAGGGGGGACCTTCCAT; | (SEQ ID NO: 105) |
| GAGAAGGGGGGACCTTCCAT; | (SEQ ID NO: 106) |
| GAGAAGGGGGGACCTTGGAT; | (SEQ ID NO: 107) |
| GAGAAGGGGGGACCTTCCAT; | (SEQ ID NO: 108) |
| GAGAAGGGGGGACCTTCCAT; | (SEQ ID NO: 109) |
| GAGAAGGGGCCAGCACTGAT; | (SEQ ID NO: 110) |
| TCCATGTGGGGCCTGATGCT; | (SEQ ID NO: 111) |
| TCCATGTGGGGCCTGATGCT; | (SEQ ID NO: 112) |
| TCCATGAGGGGCCTGATGCT; | (SEQ ID NO: 113) |
| TCCATGTGGGGCCTGCTGAT; | (SEQ ID NO: 114) |
| ATGGACTCTCCGGGGTTCTC; | (SEQ ID NO: 115) |
| ATGGAAGGTCCGGGGTTCTC; | (SEQ ID NO: 116) |
| ATGGACTCTGGAGGGGTCTC; | (SEQ ID NO: 117) |
| ATGGAGGCTCCATGGGGCTC; | (SEQ ID NO: 118) |
| ATGGACTCTGGGGGGTTCTC; | (SEQ ID NO: 119) |
| ATGGACTCTGGGGGGTTCTC; | (SEQ ID NO: 120) |
| TCCATGTGGGTGGGGATGCT; | (SEQ ID NO: 121) |
| TCCATGCGGGTGGGGATGCT; | (SEQ ID NO: 122) |
| TCCATGGGGGTCCTGATGCT; | (SEQ ID NO: 123) |
| TCCATGGGGGTCCTGATGCT; | (SEQ ID NO: 124) |
| TCCATGTGGGGCCTGATGCT; | (SEQ ID NO: 125) |
| TCCATGTGGGGCCTGATGCT; | (SEQ ID NO: 126) |
| TCCATGGGGTCCCTGATGCT; | (SEQ ID NO: 127) |
| TCCATGGGGTGCCTGATGCT; | (SEQ ID NO: 128) |
| TCCATGGGGTTCCTGATGCT; | (SEQ ID NO: 129) |
| TCCATGGGGTCCCTGATGCT; | (SEQ ID NO: 130) |
| TCCATCGGGGGCCTGATGCT; | (SEQ ID NO: 131) |
| GCTAGAGGGAGTGT; | (SEQ ID NO: 132) |
| GGGGGGGGGGGGGGGGGGGG; | (SEQ ID NO: 133) |
| ACTGACAGACTGACAGACTGA; | (SEQ ID NO: 134) |
| AGTGACAGACAGACACACTGA; | (SEQ ID NO: 135) |
| ACTGACAGACTGATAGACCCA; | (SEQ ID NO: 136) |
| AGTGAGAGACTGCAAGACTGA; | (SEQ ID NO: 137) |
| AATGCCAGTCCGACAGGCTGA; | (SEQ ID NO: 138) |
| CCAGAACAGAAGCAATGGATG; | (SEQ ID NO: 139) |
| CCTGAACAGAAGCCATGGATG; | (SEQ ID NO: 140) |
| GCAGAACAGAAGACATGGATG; | (SEQ ID NO: 141) |
| CCACAACACAAGCAATGGATA; | (SEQ ID NO: 142) |
| AAGCTAGCCAGCTAGCTAGCA; | (SEQ ID NO: 143) |
| CAGCTAGCCACCTAGCTAGCA; | (SEQ ID NO: 144) |
| AAGCTAGGCAGCTAACTAGCA; | (SEQ ID NO: 145) |
| GAGCTAGCAAGCTAGCTAGGA; | (SEQ ID NO: 146) |

For use in the instant invention, the immunostimulatory nucleic acids may be synthesized de novo using any of a number of procedures well known in the art. Such compounds are referred to as “synthetic” nucleic acids. For example, the b-cyanoethyl phosphoramidite method (Beaucage, S. L., and Caruthers, M. H., Tet. Let. 22:1859, 1981); nucleoside H-phosphonate method (Garegg et al., Tet. Let. 27:4051-4054, 1986; Froehler et al., Nucl. Acid. Res. 14:5399-5407, 1986,; Garegg et al., Tet. Let. 27:4055-4058, 1986, Gaffney et al., Tet. Let. 29:2619-2622,1988). These chemistries can be performed by a variety of automated oligonucleotide synthesizers available in the market. These nucleic acids are referred to as synthetic nucleic acids. Alternatively, immunostimulatory nucleic acids can be produced on a large scale in plasmids, (see Sambrook, T., et al., “Molecular Cloning: A Laboratory Manual”, Cold Spring Harbor laboratory Press, New York, 1989) and separated into smaller pieces or administered whole. Nucleic acids can be prepared from existing nucleic acid sequences (e.g., genomic or cDNA) using known techniques, such as is those employing restriction enzymes, exonucleases or endonucleases. Nucleic acids prepared in this manner are referred to as isolated nucleic acids. The term “immunostimulatory nucleic acid” encompasses both synthetic and isolated immunostimulatory nucleic acids.

For use in vivo, nucleic acids are preferably relatively resistant to degradation (e.g., are stabilized). A “stabilized nucleic acid molecule” shall mean a nucleic acid molecule that is relatively resistant to in vivo degradation (e.g. via an exo- or endo-nuclease). Stabilization can be a function of length or secondary structure. Immunostimulatory nucleic acids that are tens to hundreds of kbs long are relatively resistant to in vivo degradation. For shorter immunostimulatory nucleic acids, secondary structure can stabilize and increase their effect. For example, if the 3′ end of a nucleic acid has self-complementarity to an upstream region, so that it can fold back and form a sort of stem loop structure, then the nucleic acid becomes stabilized and therefore exhibits more biological in vivo activity.

Alternatively, nucleic acid stabiliaton can be accomplished via backbone modifications. Preferred stabilized nucleic acids of the instant invention have a modified backbone. It has been demonstrated that modification of the nucleic acid backbone provides enhanced activity of the immunostimulatory nucleic acids when administered in vivo. One type of modified backbone is a phosphate backbone modification. Immunostimulatory nucleic acids, including at least two phosphorothioate linkages at the 5′ end of the oligonucleotide and multiple phosphorothioate linkages at the 3′ end, preferably 5, can in some circumstances provide maximal activity and protect the nucleic acid from degradation by intracellular exo- and endo-nucleases. Other phosphate modified nucleic acids include phosphodiester modified nucleic acids, combinations of phosphodiester and phosphorothioate nucleic acids, methylphosphonate, methylphosphorothioate, phosphorodithioate, and combinations thereof. Each of these combinations in CpG nucleic acids and their particular effects on immune cells is discussed in more detail in Issued U.S. Pat. Nos. 6,194,388; 6,207,646, and 6,239,116, the entire contents of which are hereby incorporated by reference. Although not intending to be bound by any particular theory, it is believed that these phosphate modified nucleic acids may show more stimulatory activity due to enhanced nuclease resistance, increased cellular uptake, increased protein binding, and/or altered intracellular-localization

Modified backbones such as phosphorothioates may be synthesized using automated techniques employing either phosphoramidate or H-phosphonate chemistries. Aryl-and alkyl-phosphonates can be made, e.g., as described in U.S. Pat. No. 4,469,863. Alkylphosphotri esters, in which the charged oxygen moiety is alkylated as described in U.S. Pat. No. 5,023,243 and European Patent No. 092,574, can be prepared by automated solid phase synthesis using commercially available reagents. Methods for making other DNA backbone modifications and substitutions have been described E B. and Peyman, A., Chem. Rev. 90:544, 1990; Goodchild, J., Bioconjugate Chem. 1:165, 1990).

Both phosphorothioate and phosphodiester nucleic acids containing immunostimulatory motifs are active in immune cells. However, based on the concentration needed to induce immunostimulatory nucleic acid specific effects, the nuclease resistant phosphorothioate backbone immunostimulatory nucleic acids are more potent than phosphodiester backbone immunostimulatory nucleic acids. For example, 2 μg/ml of the phosphorothioate has been shown to effect the same immune stimulation as a 90 μg/ml of the phosphodiester.

Another type of modified backbone, useful according to the invention, is a peptide nucleic acid. The backbone is composed of aminoethyl glycine and supports bases which provide the DNA character. The backbone does not include any phosphate and thus may optionally have no net charge. The lack of charge allows for stronger DNA-DNA binding because the charge repulsion between the two stands does not exist Additionally, because the backbone has an extra methylene group, the oligonucleotides are enzyme/protease resistant. Peptide nucleic acids can be purchased from various commercial sources, e.g., Perkin Elmer, or synthesized de novo.

Another class of backbone modifications include 2′-O-methylribonucleosides (2′-Ome). These types of substitutions are described extensively in the prior art and in particular with respect to their immunostimulatory properties in Zhao et al., Bioorganic and Medicinal Chemistry Letters, 1999, 9:24:3453. Zhao et al. describes methods of preparing 2′-Ome modifications to nucleic acids.

The nucleic acid molecules of the invention may include normally-occurring or synthetic purine or pyrimidine heterocyclic bases as well as modified backbones. Purine or pyrimidine heterocyclic bases include, but are not limited to, adenine, guanine, cytosine, thymidine, uracil, and inosine. Other representative heterocyclic bases are disclosed in U.S. Pat. No. 3,687,808, issued to Merigan, et al. The terms “purines” or “pyrimidines” or “bases” are used herein to refer to both naturally-occurring or synthetic purines, pyrimidines or bases.

Other stabilized nucleic acids include non-ionic DNA analogs, such as alkyl- and aryl-phosphates (in which the charged phosphonate oxygen is replaced by an alkyl or aryl group), phosphodiester and alkylphosphotriesters, in which the charged oxygen moiety is alkylated Nucleic acids which contain diol such as tetraethyleneglycol or hexaethyleneglycol, at either or both termini have also been shown to be substantially resistant to nuclease degradation.

The immunostimulatory nucleic acids having backbone modifications useful according to the invention in some embodiments are S- or R-chiral immunostimulatory nucleic acids. An “S chiral immunostimulatory nucleic acid” as used herein is an immunostimulatory nucleic acid wherein at least two nucleotides have a backbone modification forming a chiral center and wherein at least 75% of the chiral centers have S chirality. An “R chiral immunostimulatory nucleic acid” as used herein is an immunostimulatory nucleic acid wherein at least two nucleotides have a backbone modification forming a chiral center and wherein at least 75% of the chiral centers have R chirality. The backbone modification may be any type of modification that forms a chiral center. The modifications include but are not limited to phosphorothioate, methylphosphonate, methylphosphorothioate, phosphorodithioate, 2′-Ome and combinations thereof.

The chiral immunostimulatory nucleic acids must have at least two nucleotides within the nucleic acid that have a backbone modification. All or less than all of the nucleotides in the nucleic acid, however, may have a modified backbone. Of the nucleotides having a modified backbone (referred to as chiral centers), at least 75% of the have a single chirality, S or R. Thus, less than all of the chiral centers may have S or R chirality as long as at least 75% of the chiral centers have S or R chirality. In some embodiments at least 80%, 85%, 90%, 95%, or 100% of the chiral centers have S or R chirality. In other embodiments at least 80%, 85%, 90%, 95%, or 100% of the nucleotides have backbone modifications.

The S- and R-chiral immunostimulatorynucleic acids may be prepared by any method known in the art for producing chirally pure oligonucleotides. Stec et al teach methods for producing stereopure phosphorothioate oligodeoxynucleotides using an oxathiaphospholane. (Stec, W. J., et al., 1995, J Am. Chem. Soc, 117:12019). Other methods for making chirally pure oligonucleotides have been described by companies such as ISIS Pharmaceuticals. U.S. patents which disclose methods for generating stereopure oligonucleotides include U.S. Pat. Nos. 5,883,237, 5,837,856, 5,599,797, 5,512,668, 5,856,465, 5,359,052, 5,506,212, 5,521,302 and 5,212,295, each of which is hereby incorporated by reference in its entirety.

As used herein, administration of an immunostimulatory nucleic acid is intended to embrace the administration of one or more immunostimulatory nucleic acids which may or may not differ in of their profile, sequence, backbone modifications and biological effect. As an example, CpG nucleic acids and poly-G nucleic acids may be administered to a single subject. In another example, a plurality of CpG nucleic acids which differ in nucleotide sequence may also be administered to a subject.

The therapeutic formulations of the invention are oil-in-water emulsions. As used herein the term oil-in-water emulsion refers to a fluid composed of a heterogeneous mixture of minute drops of oil suspended in water. Oil-in-water emulsions are well known in the art. One preferred oil-in-water emulsion is sold under the trademark name EMULSIGEN™ (sold by MPV Laboratories, Nebraska, U.S.A).

The ten “effective amount” of an immunostimulatory nucleic acid refers to the amount necessary or sufficient to realize a desired biologic effect. For example, an effective amount of an immunostimulatory nucleic acid could be that amount necessary to cause activation of the immune system, resulting potentially in the development of an antigen specific immune response. According to some aspects of the invention, an effective amount is that amount of an immunostimulatory nucleic acid and that amount of a therapeutic formulation, which when combined or co-administered, results in a synergistic response to the cancer or infectious agent, either in the prevention or the treatment of the cancer or infectious disease. A synergistic amount is that amount which produces a response that is greater than the sum of the individual effects of either the immunostimulatory nucleic acid and the therapeutic formulation alone. For example, a synergistic combination of an immunostimulatory nucleic acid and a therapeutic formulation provides a biological effect which is greater than the combined biological effect which could have been achieved using each of the components (i.e., the nucleic acid and the medicament) separately. The biological effect may be the amelioration and or absolute elimination of symptoms resulting from the cancer or infectious disease. In another embodiment, the biological effect is the complete abrogation of the cancer or infectious disease, as evidenced for example, by the absence of a tumor or a biopsy or blood smear which is free of cancer cells.

The effective amount of immunostimulatory nucleic acid necessary to synergize with a therapeutic formulation in the treatment of a cancer or infectious disease or in the reduction of the risk of developing a cancer or infectious disease may vary depending upon the sequence of the immunostimulatory nucleic acid, the backbone constituents of the nucleic acid, and the mode of delivery of the nucleic acid. The effective amount for any particular application can also vary depending on such factors as the disease being treated, the particular immunostimulatory nucleic acid being administered (e.g. the nature, Number or location of immunostimulatory motif in the nucleic acid), the size of the subject, or the severity of the disease or condition. One of ordinary skill in the art can empirically determine the effective amount of a particular in stimulatory nucleic acid and therapeutic formulation combination without necessitating undue experimentation. Combined with the teachings provided herein, by choosing among the various active compounds and weighing factors such as potency, relative bioavailability, patient body weight, severity of adverse side-effects and preferred mode of administration, an effective prophylactic or therapeutic treatment regimen can be planned which does not cause substantial toxicity and yet is entirely effective to treat the particular subject.

In some embodiments, the immunostimulatory nucleic acids are administered in an effective amount to stimulate or induce a Th1 immune response, or a Th2 immune response, or a general immune response. An effective amount to stimulate a Th1 immune response may be defined as that amount which stimulates the production of one or more Th1-type cytokines such as interleukin 2 (IL-2), IL-12, tumor necrosis factor (TNFA) and interferon gamma (IFN-γ), and/or production of one or more Th1 -type antibodies. An effective amount to stimulate a Th2 immune response, on the other hand, may be defined as that amount which stimulates the production of one or more Th2-type cytokines such as IL4, IL-5, IL-6, IL-9, IL-10 and IL-13, and/or the production of one or more Th2-type antibodies.

In some embodiments of the invention, the immunostimulatory nucleic acid is administered in an effective amount for preventing bacterial, viral or fugal infection Immunostimulatory nucleic acids are known to be useful for preventing bacterial and viral infections.

In some instances, a sub-therapeutic dosage of the antigen is used in the treatment of a subject having, or at risk of developing, cancer or infectious disease. As an example, it has been discovered according to the invention, that when the antigen is used together with the immunostimulatory nucleic acid, the antigen can be administered in a sub-therapeutic dose and still produce a desirable therapeutic result. A “sub-therapeutic dose” as used herein refers to a dosage which is less than that dosage which would produce a therapeutic result in the subject if administered in the absence of the other agent Thus, the sub-therapeutic dose of an antigen is one which, alone or in combination with a conventional adjuvant such as alum, would not produce the desired therapeutic result in the subject in the absence of the administration of the immunostimulatory nucleic acid. Therapeutic doses of antigens are well known in the field of vaccination. These dosages have been extensively described in references relied upon by the medical profession as guidance for vacination. Therapeutic dosages of immunostimulatory nucleic acids have also been described in the art and methods for identifying therapeutic dosages in subjects are described in more detail herein.

For any compound described herein a therapeutically effective amount can be initially determined from cell culture assays. In particular, the effective amount of immunostimulatory nucleic acid can be determined using in vitro stimulation assays. The stimulation index of the immunostimulatory nucleic acid can be compared to that of previously tested immunostimulatory acids. The stimulation index can be used to determine an effective amount of the particular oligonucleotide for the particular subject, and the dosage can be adjusted upwards or downwards to achieve the desired levels in the subject.

Therapeutically effective amounts can also be determined in animal studies. For instance, the effective amount of immunostimulatory nucleic acid and therapeutic formulation to induce a synergistic response can be assessed using in vivo assays of tumor regression and/or prevention of tumor formation Relevant animal models include assays in which malignant cells are injected into the animal subjects, usually in a defined site. Generally, a range of immunostimulatory nucleic acid doses are administered into the animal along with a range of therapeutic formulation doses. Inhibition of the growth of a tumor following the injection of the malignant cells is indicative of the ability to reduce the risk of developing a cancer. Inhibition of further growth (or reduction in size) of a pre-existing tumor is indicative of the ability to treat the cancer. Mice which have been modified to have human immune system elements can be used as recipients of human cancer cell lines to determine the effective amount of the synergistic combination.

A therapeutically effective dose can also be determined from human data for immunostimulatory nucleic acids which have been tested in humans (human clinical trials have been initiated) and for compounds which are known to exhibit similar pharmacological activities, such as other adjuvants, e.g., LT and other antigens for vaccination purposes.

The applied dose of both the immunostimulatory nucleic acid and the therapeutic formulation can be adjusted based on the relative bioavailability and potency of the administered compounds, including the adjuvants used. Adjusting the dose to achieve maximal efficacy based on the methods described above and other methods are well within the capabilities of the ordinarily skilled artisan.

Subject doses of the compounds described herein typically range from about 0.1 μg to 10,000 mg, more typically from about 1 μg/day to 8000 mg, and most typically from about 10 μg to 100 μg. Stated in terms of subject body weight, typical dosages range from about 0.1 μg to 20 mg/kg/day, more typically from about 1 to 10 mg/kg/day, and most typically from about 1 to 5 mg/kg/day.

In other embodiments of the invention, the immunostimulatory nucleic acid is administered on a routine schedule. A “routine schedule” as used herein, refers to a predetermined designated period of time. The routine schedule may encompass periods of time which are identical or which differ in-length, as long as the schedule is predetermined. For instance, the routine schedule may involve administration of the immunostimulatory nucleic acid on a daily basis, every two days, every three days, every four days, every five days, every six days, a weekly basis, a monthly basis or any set number of days or weeks there-between, every two months, three months, four months, five months, six months, seven months, eight months, nine months, ten months, eleven months, twelve months, etc. Alternatively, the predetermined routine schedule may involve administration of the immunostimulatory nucleic acid on a daily basis for the first week, followed by a monthly basis for several months, and then every three months after that Any particular combination would be covered by the routine schedule as long as it is determined ahead of time that the appropriate schedule involves administration on a certain day.

The immunostimulatory nucleic acids may be delivered to the subject in the form of a plasmid vector. In some embodiments, one plasmid vector could include both the immunostimulatory nucleic acid and a nucleic acid encoding an antigen. In other embodiments, separate plasmids could be used. In yet other embodiments, no plasmids could be used.

The immunostimulatory nucleic acid and the therapeutic formulation may be administered alone (e.g. in saline or buffer) or using any delivery vectors known in the art For instance the following delivery vehicles have, been described: cochleates (Gould-Fogerite et al., 1994, 1996); Emulsomes (Vancott et al., 1998, Lowell et al., 1997); ISCOMs (Mowat et al., 1993, Carlsson et al., 1991, Hu et., 1998, Morein et al., 1999); liposomes (Childers et al., 1999, Michalek et al., 1989; 1992,.de Haan 1995a, 1995b); live bacterial vectors (e.g., Salmonella, Escherichia coli, Bacillus calmatte-guerin, Shigella, Lactobacillus) (Hone et al. 1996, Pouwels et al., 1998, Chatfield et al., 1993, Stover et al., 1991, Nugent et al., 1998); live viral vectors (e.g., Vaccinia, adenovirus, Herpes Simplex) (Gallichan et al., 1993, 1995, Moss et al., 1996, Nugent et al., 1998, Flexner et al., 1988, Morrow et al., 1999); microspheres (Gupta et al., 1998, Jones et al., 1996, Maloy et al., 1994, Moore et al., 1995, O'Hagan et al., 1994, Eldridge et al., 1989); nucleic acid vaccines (Fynan et al., 1993, Kuklin et al., 1997, Sasaki et al., 1998, Okada et al., 1997, Ishii et al., 1997); polymers (e.g. carboxymethylcellulose, chitosan) (Hamajima et. al., 1998, Jabbal-Gill et al., 1998); polymer rings (Wyatt et al., 1998); proteosomes (Vancott et al., 1998, Lowell et al., 1988, 1996, 1997); sodium fluoride (Hashi et al., 1998); transgenic plants (Tacket et al., 1998, Mason et al., 1998, Haq et al, 1995); virosomes (Gluck et al., 1992, Mengiardi et al., 1995, Cryz et al., 1998); and, virus-like particles (Jiang et al., 1999, Leibl et al., 1998).

The immunostimulatory nucleic acid may be combined with additional therapeutic agents such as cytolines to enhance immune responses even further. The immunostimulatory nucleic acid and other therapeutic agent may be administered simultaneously or sequentially. When the other therapeutic agents are administered simultaneously they can be admni istered in the same or separate formulations, but are administered at the same time. The administration of the other therapeutic agents and the immunostimulatory nucleic acid may also be temporally separated, meaning that the therapeutic agents are administered at a different time, either before or after, the adininistration of the immluostimulatory nucleic acid. The separation in time between the administration of these compounds may be a matter of minutes or it may be longer. Other therapeutic agents include but are not limited to cytokines, immunotherapeutic antibodies, antigens, etc.

Immune responses can also be induced or augmented by the coadminlstration or co-linear expression of cytokines or co-stimulatory molecules with the immunostimulatory nucleic acids. The cytokines may be adminiered directly with immunostiunulatory nucleic acids or may be administered in the form of a nucleic acid vector that encodes the cytokine, such that the cytoline can be expressed in vivo. In one embodiment, the cytokine is administered in the form of a plasmid expression vector. The term “cytokine” is used as a generic name for a diverse group of soluble proteins and peptides which act as humoral regulators at nano- to picomolar concentrations and which, either under normal or pathological conditions, modulate the functional activities of individual cells and tissues. These proteins also mediate interactions between cells directly and regulate processes taking place in the extracellular environment. Cytokines also are central in directing the T cell response. Examples of cytokines include, but are not limited to IL-1, IL-2, IL-4, IL-5, IL-6, IL-7, IL-10, IL-12, IL-15, IL-18, granulocyte-macrophage colony stimulating factor (GM-CSF), granulocyte colony stimulating factor (GCSF), interferon-γ (IFN-γ), IFN-α, tumor necrosis factor (TNF), TGF-β, FLT-3 ligand, and CD40 ligand. In some embodiments, the cytokine is a Th1 cytokine. In still other embodiments, the cytolide is a Th2 cytokine. In other embodiments a cytokine is not administer in combination with the immunostimulatory nucleic acid.

In other aspects, the invention relates to kits. One kit of the invention includes a container housing an immunostimulatory nucleic acid and a container housing an oil-in-water emulsion and instructions for timing of administration of the immunosemlatory nucleic acid and the oil-in-water emulsion. Another kit of the invention includes a container housing an immunostimulatory nucleic acid and instructions for timing of administration of the immunostimulatory nucleic acid. Optionally the kit may also include an antigen, housed in a separate container or formulated with the immunostimulatory nucleic acid or therapeutic formulation. Optionally the antigen may be in a sustained release device. A sustained release vehicle is used here in accordance with its prior art meaning of any device which slowly releases the antigen.

Such systems can avoid repeated administrations of the compounds, increasing convenience to the subject and the physician. Many types of release delivery systems are available and known to those of ordinary skill in the art. They include polymer base systems such as poly(lactide-glycolide), copolyoxalates, polycaprolactones, polyesteramides, polyorthoesters, polyhydroxybutyric acid, and polyanhydrides. Micro capsules of the foregoing polymers containing drugs are described in, for example, U.S. Pat. No. 5,075,109. Delivery systems also include non-polymer systems that are: lipids including sterols such as cholesterol, cholesterol esters and fatty acids or neutral fats such as mono(di- and tri-glycerides; hydrogel release systems; sylastic systems; peptide based systems; wax coatings; compressed tablets using conventional binders and excipients; partially fused implants; and the like. Specific examples include, but are not limited to: (a) erosional systems in which an agent of the invention is contained in a form within a matrix such as those described in U.S. Pat. Nos. 4,452,775, 4,675,189, and 5,736,152, and (b) diffusional systems in which an active component permeates at a controlled rate from a polymer such as described in U.S. Pat. Nos. 3,854,480, 5,133974 and 5,407,686. In addition, pump based hardware delivery systems can be used, some of which are adapted for implantation.

The formulations such as the oil-in-water-emulsion are housed in at least one container. The container may be a single container housing all of the formulation together or it may be multiple containers or chambers housing individual- dosages, such as a blister pack. The kit also has instructions for timing of administration of the therapeutic formulation. The instructions would direct the subject having cancer or at risk of cancer to take the therapeutic formulation at the appropriate time. For instance, the appropriate time for delivery of the medicament may be as the symptoms occur. Alternatively, the appropriate time for administration of the medicament may be on a routine schedule such as monthly or yearly.

The pharmaceutical compositions of the invention contain an effective amount of an immunostimulatory nucleic acid and therapeutic formulation optionally included in a pharmaceutically-acceptable carrier. The term “pharmaceutically-acceptable carrier” means one or more compatible solid or liquid filler, dilutants or encapsulating substances which are suitable for administration to a human or other vertebrate animal. The term “carrier” denotes an organic or inorganic ingredient, natural or synthetic, with which the active ingredient is combined to facilitate the application The components of the pharmaceutical compositions also are capable of being commingled with the compounds of the present invention, and with each other, in a manner such that there is no interaction which would substantially impair the desired pharmaceutical efficiency.

The immunostimulatory nucleic acid and therapeutic formulation may be administered per se (neat) or in the form of a pharmaceutically acceptable salt When used in medicine the salts should be pharmaceutically acceptable, but non-pharmaceutically acceptable salts may conveniently be used to prepare pharmaceutically acceptable salts thereof. Such salts include, but are not limited to, those prepared from the following acids: hydrochloric, hydrobromic, sulphuric, nitric, phosphoric, maleic, acetic, salicylic, p-toluene sulphonic, tartaric, citric, methane sulphonic, formic, malonic, succinic, naphthalene-2-sulphonic, and benzene sulphonic. Also, such salts can be prepared as alkaline metal or aquiline earth salts, such as sodium, potassium or calcium salts of the carboxylic acid group.

Suitable buffering agents include: acetic acid and a salt (1-2% w/v); citric acid and a salt (1-3% w/v); boric acid and a salt (0.5-2.5% w/v); and phosphoric acid and a salt (0.8-2% w/v). Suitable preservatives include benzalkonium chloride (0.003-0.03% w/v); chlorobutanol (0.3-0.9% w/v); parabens (0.01-0.25% w/v) and thimerosal (0.0040.02% w/v).

Pharmaceutical formulations for parenteral administration include aqueous solutions of the active compounds in water-soluble form. Additionally, suspensions of the active compounds may be prepared as appropiate oily injection suspensionls. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol or dextran. Optionally, the suspension may also contain suitable stabilizes or agents which increase the solubility of the compounds to allow for the preparation of highly concentrated solutions. Another suitable compound for sustained release delivery is GELFOAM, a commercially available product consisting of modified collagen fibers.

Alternatively, the active compounds may be in powder form for constitution with a suitable vehicle, e.g., sterile pyrogen-free water, before use.

The pharmaceutical compositions also may comprise suitable solid or gel phase carriers or excipients. Examples of such carriers or excipients include but are not limited to calcium carbonate, calcium phosphate, various sugars, starches, cellulose derivatives, gelatin, and polymers such as polyethylene glycols.

The immunostimulatory nucleic acid and therapeutic formulations can be administered on fixed schedules or in different temporal relationships to one another. The various combinations have many advantages over the prior art methods.

Immunostimulatory nucleic acid and therapeutic formulation may be administered by any ordinary route for administering medications. Depending upon the type of disorder to be treated, immunostimulatory nucleic acids and therapeutic formulations may be inhaled, ingested or administered by systemic routes. Systemic routes include oral and parenteral. Inhaled medications are preferred in some embodiments because of the direct delivery to the lung, particularly in the treatment of respiratory disease or lung cancer. Several types of metered dose inhalers are regularly used for administration by inhalation. These types of devices include metered dose inhalers (MDI), breath-actuated MDI, dry powder inhaler (DPI), spacer/holding chambers in combination with MDI, and nebulizers. Preferred routes of administration include but are not limited to oral, parenteral, intramuscular, intranasal, intratracheal, intrathecal, intravenous, inhalation, ocular, vaginal, and rectal.

For use in therapy, an effective amount of the immunostimulatory nucleic acid and therapeutic formulation can be administered to a subject by any mode that delivers the nucleic acid to the affected organ or tissue, or alternatively to the immune system. “Administering” the pharmaceutical composition of the present invention may be accomplished by any means known to the skilled artis. Preferred routes of administration include but are not limited to oral, parenteral, intramuscular, subcutaneous, intranasal, intratracheal, inhalation, ocular, vaginal, and rectal.

For oral administration, the compounds (i.e., immunostimulatory nucleic acids, therapeutic formulations, and the other therapeutic agents) may be formulated readily by combining the active compound(s) with pharmaceutically acceptable carriers well known in the art. Such carriers enable the compounds of the invention to be formulated as tablets, pills, degrees, capsules, liquids, gels, syrups, slurries, suspensions and the like, for oral ingestion by a subject to be treated. Pharmaceutical preparations for oral use can be obtained as solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the cross-linked polyvinyl pyrrolidone, agar, or alginic acid or a salt thereof such as sodium alginate. Optionally the oral formulations may also be formulated in saline or buffers for neutralizing internal acid conditions or may be administered without any carriers.

Dragee cores are provided with suitable coatings. For this purpose, concentrated sugar solutions may be used, which may optionally contain gum arabic, talc, polyvinyl pyrrolidone, carbonyl gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compound doses.

Pharmaceutical preparations which can be used orally include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. Microspheres formulated for oral administration may also be used. Such microspheres have been well defined in the art. All formulations for oral administration should be in dosages suitable for such administration.

For buccal administration, the compositions may take the form of tablets or lozenges formulated in conventional manner.

For administration by inhalation, the compounds for use according to the present invention may be conveniently delivered in the form of an aerosol spray presentation from pressurized packs or a nebulizer, with the use of a suitable propellant, e.g., dichlorofluoromethoxy, trichlorofluoromethane, dichlorotetrafluoroethane, carbon dioxide or other suitable gas. In the case of a pressurized aerosol the dosage unit may be determined by providing a valve to deliver a metered amount Capsules and cartridges of e.g. gelatin for use in an inhaler or insulator may be formulated containing a powder mix of the compound and a suitable powder base such as lactose or starch. Techniques for preparing aerosol delivery systems are well known to those of skill in the art. Generally, such systems should utilize components which will not significantly impair the biological properties of the therapeutic, such as the immunostimulatory capacity of the nucleic acids (see, for example, Sciarra and Cutie, “Aerosols,” in Remington's Pharmaceutical Sciences, 18th edition, 1990, pp 1694-1712; incorporated by reference). Those of skill in the art can readily determine the various parameters and conditions for producing aerosols without resort to undue experimentation.

The compounds, when it is desirable to deliver them systemically, may be formulated for parenteral administration by injection, e.g., by bolus injection or continuous infusion. Formulations for injection may be presented in unit dosage form, e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents.

In still other embodiments of the invention, the immunostimulatory nucleic acids are provided in the intravenous solutions, bags and/or tubing used to deliver transfusions into cancer patients. The immunostimulatory nucleic acids may be introduced into an intravenous solution which is administered to the subject prior to receiving the transfusion,.or it may be introduced into the blood transfusion itself (i.e., the suspension of red blood cells or platelets). Alternatively, the intravenous bags and tubing may be themselves be coated on their internal surfaces with immunostimulatory nucleic acids, or they may be impregnated with immunostimulatory nucleic acids during manufacture. Methods for manufacture of intravenous systems for the delivery of biologically active materials are known in the art. Examples include those described in U.S. Pat. Nos.: 4,973,307, and 5,250,028, issued to Alza, Corp.

The compounds may also be formulated in rectal or vaginal compositions such as suppositories or retention enemas, e.g., containing conventional suppository bases such as cocoa butter or other glycerides.

In addition to the formulations described previously, the compounds may also be formulated as a depot preparation. Such long acting formulations may be formulated with suitable polymeric or hydrophobic material (for example as an emulsion in an acceptable oil) or ion exchange resins, or as sparingly soluble derivatives, for example, as a sparingly soluble salt.

Suitable liquid or solid pharmaceutical preparation forms are, for example, aqueous or saline solutions for inhalation, microencapsulated, encochleated, coated onto microscopic gold particles, contained in liposomes, nebulized, aerosols, pellets for implantation into the skin, or dried onto a sharp object to be scratched into the skin The pharmaceutical compositions also include granules, powders, tablets, coated tablets, (micro)capsules, suppositories, syrups, emulsions, suspensions, creams, drops or preparations with protracted release of active compounds, in whose preparation excipients and additives and/or auxiliaries such as disintegrates, binders, coating agents, swelling agents, lubricants, flavorings, sweeteners or solubilizers are customarily used as described above. The pharmaceutical compositions are suitable for use in a variety of drug delivery systems. For a brief review of methods for drug delivery, see Langer, Science 249:1527-1533, 1990, which is incorporated herein by reference.

The present invention is further illustrated by the following Examples, which in no way should be construed as further limiting.

### EXAMPLE 1

### CpG in Combination with EMULSIGEN™:

The experiments were performed to test the immunogenicity and protective efficacy of a bovine herpesvirus-1 (BHV-1) subunit vaccine co-adjuvanted with EMULSIGEN™ (Em) and a CpG ODN in cattle. A truncated version of BHV-1 glycoprotein D (tgD) coadjuvanted with Em and CpG ODN at concentrations of 25, 2.5 or 0.25 mg/dose produced a stronger and more balanced Th1/Th2 immune response, higher serum neutralization antibodies and greater protection following BHV-1 challenge, compared to tgD adjuvanted with VSA3, Em, or CpG ODN alone. Furthermore, tgD co-adjuvanted with Em and 25 mg of a non-CpG ODN/dose produced comparable levels of immunity to Em alone and lower than the CpG ODN/Em combinations.

Materials and Methods

Cells and Virus: Strins P8-2 and 108 of BHV-1 were propagated in Madin Darby bovine kidney MDBK) cells as described previously (van Drunen Little-van den Hurk S., J. et al. 1994. A subunit gIV vaccine, produced by transfected mammalian cells in culture, induces mucosal immunity against bovine herpesvirus-1 in cattle. Vaccine 12:1295-1302.). Strain 108 was used for challenging animals, whereas for stimulation of in vitro proliferation of PBMC, strain P8-2 was used

Production, processing and purification of BHV-1 tgD: A truncated version of BHV-1 gD (tgD) was constructed by terminating the protein at amino acid 355, immediately upstream of the transmembrane anchor. It was expressed in MDBK cells under regulation of the bovine heat shock 70 A (hsp 70) gene promoter (Kowalski, J et al 1993. Heat-shock promoter-driven syntheses of secreted bovine herpesvirus glycoproteins in transfected cells. Vaccine 11:1100-1107). Truncated gD was produced, processed and purified as described elsewhere (van Drunen Little-van den Hurk, S., J. et at 1994. A. subunit gIV vaccine, produced by transfected mammalian cells in culture, induces mucosal immunity against bovine herpesvirus-1 in cattle. Vaccine 12:1295-1302).

CpG and non-CpG ODN: Unmethylated CpG dinucleotides in a synthetic oligodeoxynucleotide (ODN) preparation (Qiagen, Milden, Germany) were used either as adjuvant or as co-adjuvant in this study. The CpG ODN used was ODN 2007 (TCGTCGTTGTCGTTTTGTCGTT; CpG motifs are underlined). To determine whether immune responses were induced by the CpG dinucleotides, we also used a non-CpG ODN; 2041 (CTGGTCTTTCTGGTTTTTTTCTGG) (Qiagen). The CpG and non-CpG ODN were phosphorothioate modified to increase resistance to nuclease degradation Kuhnle, G., A et al 1998. The class II membrane glycoprotein G of bovine respiratory syncytia virus, expressed from a synthetic open reading frame, is incorporated into virions of recombinant bovine herpesvirus 1. J. Virol. 72:3804-3811).

Immunization: Eight groups of seven, nine month-old, BHV-1-seronegative Angus and Hereford cross calves were immunized subcutaneously with 50 μg BHV-1 tgD adjuvanted with either 30% vol/vol EMULSIGEN™ (Em) (MVP Laboratories, Nebraska, U.S.A), 30% vol/vol VSA3 (Em containing 24 mM dimethyldioctadecylammonium bromide [DDA]), 25 mg of CpG ODN (CpG), a combination of 30% Em and 25 (high), 2.5 (medium) or 0.25 (low) mg CpG ODN (H CpG/Em, M CpG/Em, L CpG/Em respectively), or with a combination of Em and 25 mg non-CpG ODN (non-CpG/Em). The vaccines were administered subcutaneously in a 2 ml volume. A placebo group of calves was immunized with 2 ml PBS only. Thirty-nine days later, the animals were re-immunized and then challenged 2 weeks after the secondary immunization (Day 53 of vaccination).

Experimental challenge and clinical evaluation: Five weeks after secondary immunization, animals were transported into an isolation pen weighed and examined clinically. The calves were then individually exposed for 4 min to an aerosol of 10⁷ PFU of BHV-1 as previously described (Loehr, B. I., et al. 2000. Gene gun-mediated DNA immunization primes development of mucosal immunity against bovine herpesvirus 1 in cattle. J. Virol. 74:6077-6086; van Drunen Little-van den Hurk, S., et al. 1990. Epitope specificity of the protective immune response induced by individual bovine herpesvirus-1 glycoproteins. Vaccine 8:358-368). Following challenge, the calves were weighed daily. Furthermore, they were clinically evaluated for 11 consecutive days. Clinical evaluation was performed at the same time each day by a veterinarian who was blind to the vaccine status of the animals. The clinical signs evaluated included fever (rectal temperatures >40° C.), depression, rhinitis, and conjuctivitis.

Sampling and virus isolation: Animals were bled for assessment of antibody responses on days 0, 14, 39, 47, 53, 57, 61, 64 and 67 after vaccination. Blood with anticoagulant (ethylenediamine tetraacetic acid [EDTA] to a final concentration of 0.2%) was collected on days 50 and 61 for assessment of in vitro proliferation and IFN-γ production by ELISPOT and ELISA assays. Nasal tampons containing up to 5 ml of nasal fluid were collected every second day post challenge and processed the same day to measure virus shedding Virus recovered from nasal tampons was quantified by plaque titration in microtitre plates with an antibody overlay as previously described (Rouse, B. T. and L. A. Babiuk. 1974. Host responses to infectious bovine rhinotracheitis virus. III. Isolation and immunologic activities of bovine T lymphocytes. J. Immunol. 113:1391-1398).

Enzyme-linked immunosorbent assay (ELISA): In order to determine specific antibody responses before and after challenge, 96 well polystyrene microtiter plates (Immunol 2, Dynatech, Gaithersburg, Md.) were coated overnight with 0.05 μg per well of either purified tgD or purified tgB per well (Li, K, et al. 1996. Production and characterization of bovine herpesvirus 1 glycoprotein B ectodomain derivatives in an hsp70A genepromoter-based expression system. Arch Virol. 141:2019-2029). Serially diluted bovine sera, starting at 1:10 in threefold dilutions, were incubated for 2 hours at room temperature. Alkaline phosphatase (AP)-conjugated goat anti-bovine IgG (Kirkegaard & Perry Laboratories, Gaithersburg, Md.) at a dilution of 1:5,000 was used to detect bound IgG. The reaction was visualized with p-nitrophenyl phosphate (Sigma Chemical Co., Oakville, Ontario, Canada).

Immunoglobulin isotypes using enzyme-linked immunosorbent assay: In order to determine the specific IgG1 and IgG2 antibody responses of cattle immunized with tgD, polystyrene microtiter plates were coated overnight with 0.05 μg of purified tgD per well and blocked for 30 min at 37° C. with 1% heat inactivated horse serum. Serially diluted bovine sera, stating at 1:10 in threefold dilutions, were incubated overnight at 4° C. Bound antibodies were detected with monoclonal antibodies against bovine IgG1 (M-23) or IgG2 (M-37) at dilutions of 1:40,000 and 1:8000 respectively, which in turn were detected with AP-conjugated goat anti-mouse IgG (Kirkegaard & Perry Laboratories, Gaithersburg, Md.) at a dilution of 1:10,000. The reaction was visualized as for ELISA assays. Results were expressed as ratios of IgG1 to IgG2.

Virus neutralization assays: The neutralization titres of the bovine sera were determined as described previously (Babiuk, L. A., et al. 1975. Defense mechanisms against bovine herpesvirus: relationship of virus-host cell events to susceptibility to antibody complement cell lysis. Infect.Immun. 12:958-963). The titers were expressed as the reciprocal of the highest dilution of antibody that caused a 50% reduction of plaques relative to virus control.

In Vitro Proliferation of PBMC: Peripheral blood mononuclear cells (PBMC) were isolated on Ficoll-Plaque PLUS (Pharmacia, Mississauga, Ontario, Canada) and cultured in triplicate in a 96 well tissue culture plate at 3.5×10⁵ cells/well in minimum essential medium (Gibco BRL, Grand Island N.Y, U.S.A) supplemented with 10% (vol/vol) fetal bovine serum (Sigma Chemical Co), 2 mM L-glutamine (Gibco-BRL), 500 mg/ml gentamicin, 5×10⁻⁵ M 2-mercaptoethanol and 1 mg/ml dexamethasone. Cells were stimulated with gD at a final concentration of 1 μg/ml. Control cells were unstimulated. After 72 hours in culture, the cells were pulsed with [methyl-³H] thymidine (Amersham, Oakvilie, Ontario, Canada) at a concentration of 0.4 μCu/well. The cells were harvested 18 h later using a semiautomatic cell harvester (Skatron, Starling Va., U.S.A) and radioactivity was determined by scintillation counting. Proliferative responses were calculated as the means of triplicate wells and expressed as a stimulated index (SI) where SI represents counts per min in the presence of antigen divided by counts per min in the absence of antigen.

ELISPOT assays: Nitrocellulose plates (Whatman N.J., U.S.A.) were coated overnight at 4° C. with a bovine interferon-gamma (IFN-γ)-specific monoclonal antibody at a dilution of 1:400. Unbound antibody was washed off with 0.05% vol/vol PBS-Tween-20 (PBS-T) with a final wash in PBS. PBMC were isolated as for proliferation assays and cultured at 10⁶ cells/well in the presence of gD at a final concentration of 0.4 μg/ml. Control cells were cultured with media only. After 24 h, the cells were washed, resuspended in culture medium, transferred to nitrocellulose plates and incubated for a further 24 h at 37° C., after which cells were washed off with 0.05% vol/vol PBS-T. Subsequently, the plates were incubated for 2 h at RT with rabbit polyclonal antibodies against bovine IFN-γ at a dilution of 1:100 and then for 2 h at RT with biotinylated rat anti-rabbit IgG (Zymed, San Francisco, Calif., U.S.A.), followed by streptavidin-AP (GIBCO-BRL, Ontario, Canada), each at 1:1000 dilution. Bound IFN-γ was visualized using bromochloroindolyl phosphate/nitro-blue tetrazolium (BCIP/NBT) substrate tablets (Sigma Chemical Co). The plates were washed in distilled water and air dried, after which stained spots were counted under 400× magnification. The number of IFN-γ-secreting cells was expressed as the difference between the number of spots per 10⁶ cells in gD-stimulated wells and the number of spots per 10⁶ cells in control wells.

IFN-γ ELISA: Bovine PBMC were cultured as for ELISPOT assays. After 24 h, the supernatants were harvested and serially diluted in 96 well plates coated with monoclonal antibodies against bovine IFN-γ. Purified bovine IFN-γ of known concentration was used as a standard. The standard curve ranged from 2000 to 7.8 pg/ml (r>0.98). Samples and standards were assayed at eight 2-fold dilutions in PBS-T at 100 μl/well. Bound IFN-γ was detected using rabbit anti-IFN-γ IgG, which was in turn detected using AP-conjugated goat anti-rabbit IgG. The reaction was visuals described for tgD-specific antibody EISAs. The absorbance of the substrate was measured at 405 and 490 nm An ELISA reader program (Microplate manager 5, BIO RAD Laboratories, Ontario, Canada) was used to construct a standard curve and to compute the concentration of IFN-γ in the samples.

Statistical analysis: To allow for unequal distribution, all data were transformed by log transformation prior to performance of statistical analysis. Differences in serum neutralization titers, isotype ratios, in vitro proliferative responses, ELISPOT and IFN-γ ELISA data were investigated using one-way analysis of variance and Turkey's multiple comparison test. Differences in the number of animals with signs of disease among vaccine groups (temperature increase, weight loss and virus shedding) and between tgD and tgB-specific antibodies in bovine serum before and after challenge, were determined by the two-way analysis of variance and the Tukey honestly significantly different (HSD) multiple comparison test.

Results

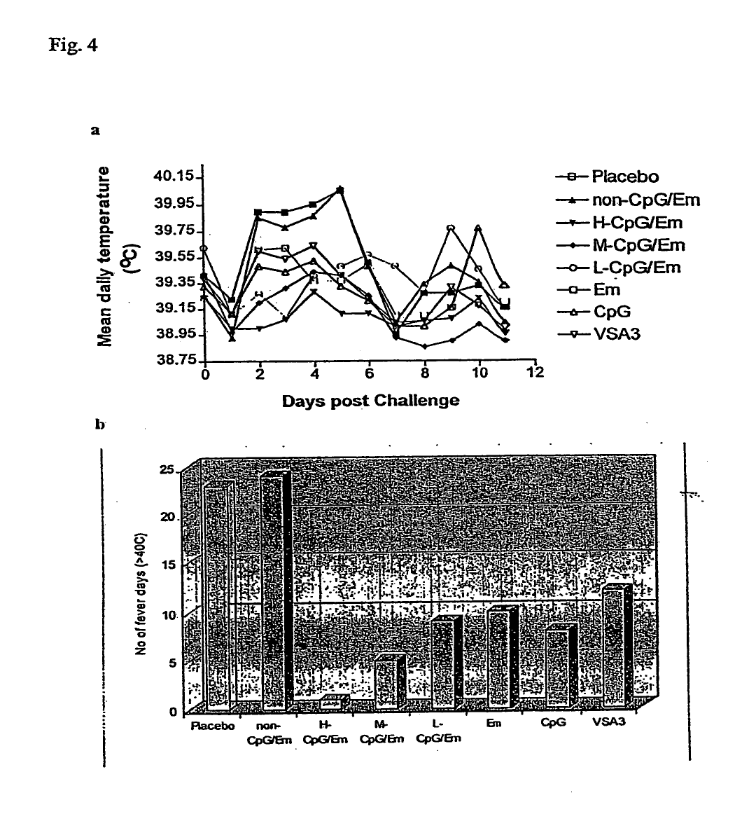

Humoral immune responses to tgD: In order to assess the adjuvant capabilities of CpG ODN, BHV-1 tgD was adjuvanted with 25 mg/dose CpG, Em or VSA3, or co-adjuvanted with Em and CpG at concentrations of 25, 2.5 or 0.25 mg/dose (H-, M- or L-CpG/Em), or with Em and 25 mg/dose of a non-CpG ODN (non-CpG/Em). With the exception of the VSA3 group, all vaccinated groups had significantly higher levels of neutralizing antibodies than the placebo group fourteen days following the primary immunization (p<0.001) (FIG. 4). Antibody levels in the H-CpG/Ex group were significantly (p<0.001) high than those of the non-CpG/Em, CpG or VSA3 groups. The antibody levels increased dramatically after secondary immunization such that on day 47 all three CpG/Em groups had significantly ([<0.001) higher titers than all other vaccine groups. This data provides evidence that the concentration of CpG ODN in the vaccines had no significant effect on the secondary immune response. Importantly, antibody titers of animals immunized with tgD co-adjuvanted with non-CpG/Em, were not significantly different from the titers of the Em group. In addition, the titers in the non-CpG/Em group were not significantly different from those of the CpG and VSA3 groups.

To determine the type of immune response generated, tgD-specific IgG1 and IgG2 antibodies in bovine serum were determined and IgG1:IgG2 ratios were measured 8 days after secondary immunization. The ratios were similar both after primary immunization and after challenge. A balanced immune response (˜1:1 ratio) was measured in the three CpG/Em groups and the CpG group, there was no statistical difference between these groups. In contrast, the Em, VSA3 and non-CpG/Em formulated vaccines produced an IgG1-biased immune response (>1600:1). The non-CpG/Em group produced a higher IgG1:IgG2 ratio than did the L-CpG/Em group. However, the non-CpG/Em group was significantly (p<0.05) different from both the M-CpG/Em and H-CpG/Em groups. There were no significant differences between the three CpG/Em groups nor between the non-CpG/Em and Em groups. In addition, all three CpG/Em groups were significantly different from both the Em (p<0.001) and VSA3 (p<0.01) groups.

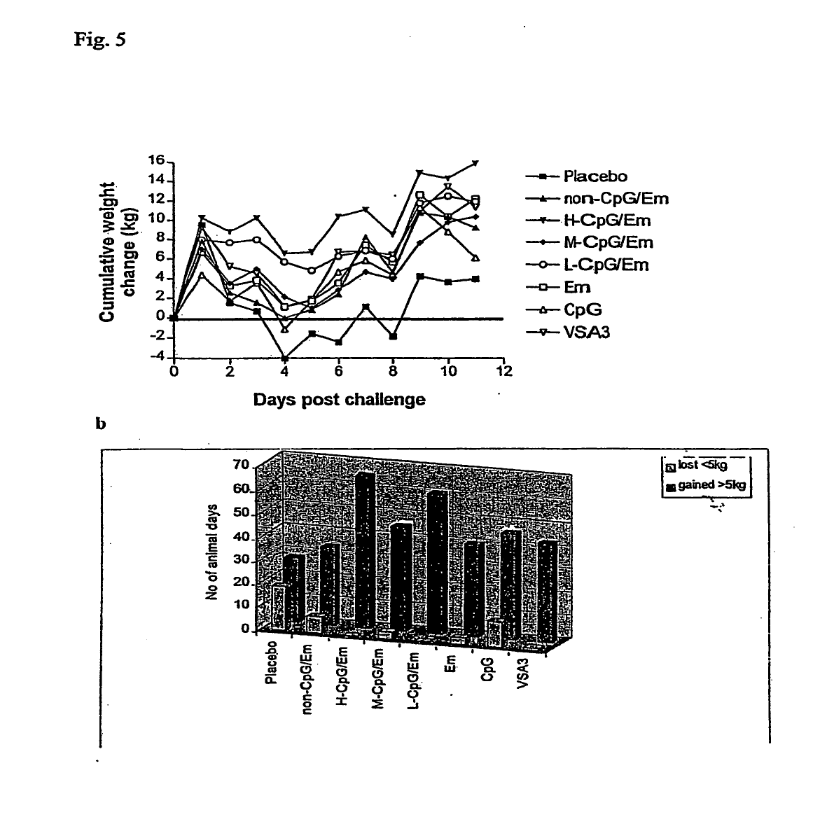

Cell-mediated immune responses to tgD: To examine cell-mediated immunity induced by the vaccinations, in vitro proliferative responses of bovine lymphocytes to BHV-1 gD were measured. Although the proliferative responses before challenge tended to be stronger in the CpG/Em vaccinated animals than in animals vaccinated with non-CpG(Em, CpG, or Em, the difference was not statistically significant (FIG. 5a). However, the proliferative responses in the H-CpG/Em and L-CpG/Em groups were significantly (p<0.05) higher than those in the VSA3 and placebo groups. To further confirm T-cell activation, production of IFN-γ was assessed. Although the numbers of IFN-7 secreting cells in the CpG/Em groups were not dependent on the concentration of CpG ODN used, they were significantly higher (p<0.001) than the number of IFN-γ secreting cells in the non-CpG/Em, Em, VSA3 and placebo groups (FIG. 5b). Following BHV-1 challenge, proliferative responses of PBMC groups were ˜2-fold stronger in the CpG/Em In in other vaccinated groups (FIG. 5b). In contrast there was no difference between the CpG/Em groups and the CpG group. The amount of IFN-γ measured before challenge in the supernatant of cultured PBMC of vaccinated animals followed a similar pattern of response to that of the ELISPOT (FIG. 5c). The CpG/Em groups were not significantly different for each other nor from the CpG or Em groups. However, the amount of IFN-γ measured in these groups was significantly higher than that measured in the placebo (p<0.01), non-CpG/Em (p<0.05) and VSA3 (p<0.01) groups. These data confirm the ability of CpG ODN even when combined with EMULSIGEN™ to induce Th1-type immune responses.

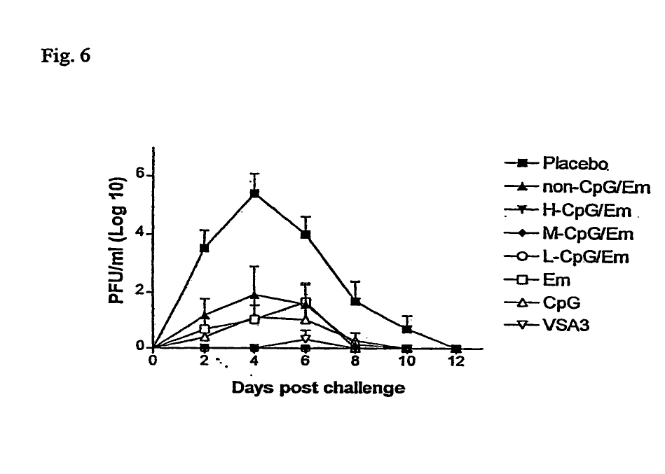

Immune responses after BHV-1 infection: An increase in the level of either serum neutralizing antibodies or antibodies specific for viral proteins after challenge is another indication of infection. Although all groups were seronegative to BHV-1 glycoprotein B (tgB) prior to challenge, antibodies against tgB in the placebo, CpG, Em, VSA3 and non-CpG/Em groups, but not in the CpG/Em groups, increased significantly (p<0.01) after challenge (FIG. 6b). Serum neutralizing titers (FIG. 4) and antibodies against tgD (FIG. 6a) in the placebo, CpG, Em, VSA3 and non-CpG/Em groups also increased significantly (p<0.005) after challenge suggesting that these groups were not entirely protected from BHV-1 infection. Although the M-CpG/Em group also exhibited some increase in both the level of serum neutralizing antibodies and antibodies against tgD after challenge, these increases were not significant. Serum neutralizing titers and antibodies against tgD in the H-CpG/Em actually decreased after challenge, while those in the M-CpG/Em group remained stable. These results suggest the induction of sterile immunity in the CpG EMULSIGEN™ formulations.

Protection from challenge with BHV-1: All animals were healthy prior to challenge. After challenge, the mean rectal temperature increased from day 2 to day 6 in all but the M- and H-CpG/Em groups, where the temperature remained ≦39.5° C. over the entire follow up period (FIG. 7). The placebo and non-pG/Em groups, exhibited the greatest increase in temperature (to ˜40.1° C. by day 6) and were significantly different from the M- and-H-CpG/Em groups (p<0.002). Although temperatures of calves in the L-CpG/Em group increased steadily to 39.5° C. by day 6 and the temperatures were significantly different (p=0.006) from those of the H-CpG)Em group, they were also different from the placebo group (p<0.001), but not from the M-CpG/Em group. The mean rectal temperature in the Em, CpG and VSA3 groups also increased to >39.4° C. on days 2 to day 4 after which it fell to <39° C.

Another assessment of morbidity is the extent of weight loss following BHV-1 infection. Whereas animals in the H- and L-CpG/Em groups experienced minimal or no loss in weight over the course of the trial, those in other groups experienced weight loss of up to 8 kg 4 days after challenge (FIG. 8).

To further determine the level of protection from BHV-1 infection, the extent of shedding from the nasal passages was assessed. Whereas animals in the CpG, Em, and non-CpG/Em groups began shedding virus on day 2 after challenge and continued to do so at least until day 8, no virus was recovered from the nasal tampons of animals in either of the CPG/Em groups (FIG. 9). Although the vaccinations had a significant (p<0.001) effect on virus shedding, the three CpG/Em groups were statistically different from only the non-CpG/Em (p=0.003) and placebo (p<0.001) groups.

The foregoing written specification is considered to be sufficient to enable one skilled in the art to practice the invention. The present invention is not to be limited in scope by examples provided, since the examples are intended as a single illustration of one aspect of the invention and other functionally equivalent embodiments are within the scope of the invention. Various modifications of the invention in addition to those shown and described herein will become apparent to those skilled in the art from the foregoing description and fall within the scope of the appended claims. The advantages and objects of the invention are not necessarily encompassed by each embodiment of the invention.

All references, patents and patent publications that are recited in this application are incorporated in their entirety herein by reference.

1. A method for reducing viral shedding in a non-human animal, comprising: administering to a non-human animal infected with a virus or at risk of viral infection, an immunostimulatory nucleic acid and an oil-in-water emulsion in an effective amount to reduce viral shedding.

2. The method of claim 1, wherein the oil-in-water emulsion is EMULSIGEN™.

3. The method of claim 1, wherein an antigen is not administered to the non-human animal.

4. The method of claim 1, further comprising administering an antigen to the non-human animal.

5. The method of claim 1, further comprising administering an antiviral agent.

6. The method of claim 5, wherein the antiviral agent is selected from the group consisting of Acemannan; Acyclovir, Acyclovir Sodium; Adefovir; Alovudine; Alvircept Sudotox; Amantadine Hydrochloride; Aranotin; Axildone; Atevirdine Mesylate; Avridine; Cidofovir; Cipamfylline; Cytarabine Hydrochloride; Delavirdine Mesylate; Desciclovir; Didanosine; Disoxaril; Edoxudine; Enviradene; Enviroxine; Famciclovir; Famotine Hydrochloride; Fiacitabine; Fialuridine; Fosarilate; Foscamet Sodium; Fosfonet Sodium; Ganciclovir; Ganciclovir Sodium; Idoxuridine; Kethoxal; Lamivudine; Lobucavir; Memotine Hydrochloride; Methisazone; Nevirapine; Penciclovir; Pirodavir; Ribavirin; Rimantadine Hydrochloride; Saquinavir Mesylate; Somantadine Hydrochloride; Sorivudine; Statolon; Stavudine; Tilorone Hydrochloride; Trifluridine; Valacyclovir Hydrochloride; Vidarabine; Vidarabine Phosphate; Vidarabine Sodium Phosphate; Viroxime; Zalcitabine; Zidovudine; and Zinviroxime.

7. The method of claim 1, wherein non-human animal is a dog, cat, horse, cow, pig, sheep, goat, primate or chicken.

8. A method for reducing tissue damage upon vaccination of a subject, comprising:
administering to a subject by an invasive route an adjuvanted vaccine and an immunostimulatory nucleic acid in an effective amount to reduce tissue damage arising from the adjuvanted vaccine, wherein the vaccine is adjuvanted with an oil-in-water emulsion.

9. The method of claim 8, wherein the oil-in-water emulsion is EMULSIGEN™.

10. The method of claim 8, wherein the invasive route is subcutaneous.

11. The method of claim 8, wherein the invasive route is intramuscular.

12. A method for inducing an immune response, comprising:
administering to a subject an oil-in-water emulsion and a CpG oligonucleotide in an effective amount to produce the immune response.

13. The method of claim 12, wherein the immune response is an antigen specific immune response.

14. The method of claim 12, further comprising administering an antigen.

15. The method of claim 12, wherein the oil-in-water emulsion is EMULSIGEN™.

16. The method of claim 12, wherein the subject has a cancer.

17. The method of claim 12, wherein the subject has an infectious disease.

18. The method of claim 12, wherein the subject is at risk of developing an infectious disease.

19. A method for reducing a dosage of antigen administered to a subject to produce an antigen specific immune response, comprising:
administering to a subject an antigen in a sub-therapeutic dosage and an immunostimulatory nucleic acid, wherein the combination of the sub-therapeutic dose of the antigen and the immunostimulatory nucleic acid produce an antigen specific immune response.

20. The method of claim 19, wherein the sub-therapeutic dose of the antigen is a dose which is at least 50% less than a minimal effective dose of antigen for producing an antigen specific immune response when the antigen is formulated with alum.

21. The method of claim 19, wherein the sub-therapeutic dose of the antigen is a dose which is at least 90% less than a minimal effective dose of antigen for producing an antigen specific immune response when the antigen is formulated with alum.

22. The method of claim 1, 8, 12, or 19, wherein the immunostimulatory nucleic acid is a CpG oligonucleotide.

23. The method of claim 22, wherein the CpG oligonucleotide is administered at regular intervals.

24. The method of claim 22, wherein the CpG oligonucleotide is administered on a weekly basis.

25. The method of claim 22, wherein the CpG oligonucleotide is administered on a daily basis.

26. The method of claim 22, wherein the CpG oligonucleotide is administered on a monthly basis.

27. The method of claim 22, wherein the CpG oligonucleotide is administered orally.

28. The method of claim 22, wherein the CpG oligonucleotide is administered by injection.

29. The method of claim 22, wherein the CpG oligonucleotide is administered through a sustained release device.

30. The method of claim 22, wherein the CpG oligonucleotide is selected from the group consisting of:

| | |
|---|---|
| 2007 | (TCGTCGTTGTCGTTTTGTCGTT); |
| 2142 | (TCGCGTGCGTTTTGTCGTTTTGACGTT); |
| 2135 | (TCGTCGTTTGTCGTTTTGTCGTT); |
| | and |
| 2216 | (ggGGGACGATCGTCgggggG). |

31. The method of claim 1, 8, 12, or 19, wherein the immunostimulatory nucleic acid is a T-rich nucleic acid.

32. The method of claim 31, wherein the T-rich nucleic acid has a sequence selected from the group consisting of SEQ ID NO: 52 through to SEQ ID NO: 57 and SEQ ID NO: 62 through to SEQ ID NO: 94.

33. The method of claim 1, 8, 12, or 19, wherein the immunostimulatory nucleic acid is a poly-G nucleic acid

34. The method of claim 33, wherein the poly-G nucleic acid has a sequence selected from the group consisting of SEQ ID-NO: 46, SEQ ID NO: 47, SEQ ID NO: 58, SEQ ID NO: 61, and SEQ ID NO: 95 through to SEQ ID NO: 133.

35. The method of claim 1, 8, 12, or 19, wherein the immunostimulatory nucleic acid has a sequence selected from the group consisting of SEQ ID NO: 1 through to SEQ ID NO: 146.

36. The method of claim 1, 8, 12, or 19, wherein the subject has a cancer selected from the group consisting of bone cancer, brain and CNS cancer, connective tissue cancer, esophageal cancer, eye cancer, Hodgkin's lymphoma, larynx cancer, oral cavity cancer, skin cancer, and testicular cancer.

37. The method of claim 1, 8, 12, or 19, wherein the immunostimulatory nucleic acid has a modified backbone.

38. The method of claim 37, wherein the modified backbone is a phosphate modified backbone.

39. The method of claim 38, wherein the phosphate modified backbone is a phosphorothioate modified backbone.

40. The method of claim 37, wherein the modified backbone is a peptide modified oligonucleotide backbone.

41. The method of claim 1, 8, 12, or 19, wherein the subject is an immunocompromised subject.

42. The method of claim 1, 8, 12, or 19, wherein the subject has an infectious disease selected from the group consisting of a viral, bacterial, fungal and parasitic infection

43. The method of claim 1, 8, 12, or 19, wherein the subject is at risk of developing an infectious disease elected from the group consisting of a viral, bacterial, fugal and is parasitic infection.

44. A composition comprising, an immunostimulatory nucleic acid and an oil-in-water emulsion.

45. The composition of claim 44, wherein the oil-in-water emulsion is EMULSIGEN™.
